(19) United States
(12) Patent Application Publication
(10) Pub. No.: US 2010/0035989 A1
(43) Pub. Date: Feb. 11, 2010

(54) COMPOSITIONS AND METHODS FOR THE TREATMENT OF MUCOSITIS

(75) Inventors: C. Eric Schwartz, Wakefield, MA (US); Per Gjorstrup, Cambridge, MA (US)

Correspondence Address: ROPES & GRAY LLP, PATENT DOCKETING 39/41, ONE INTERNATIONAL PLACE, BOSTON, MA, 02110-2624, US

(73) Assignee: Resolvyx Pharmaceuticals, Inc.

(21) Appl. No.: 12/309,430

(22) PCT Filed: Jul. 19, 2007
(86) PCT No.: PCT/US2007/016338

§ 371 Date: Sep. 4, 2009

Related U.S. Application Data
(60) Provisional application No. 60/831,866, filed on Jul. 19, 2006.

(51) Int. Cl. A61K 31/20; C07C 57/02; C07C 57/18

(52) U.S. Cl. 514/560; 562/598

(57) ABSTRACT

The invention relates to methods of treating mucositis comprising administering a compound of formula A, compound of any one of formulae 1 to 46, lipoxin compound, or oxylipin compound.

### RELATED APPLICATIONS

This application claims the benefit of priority to U.S. Provisional Patent Application No. 60/831,866, filed Jul. 19, 2006, which application is hereby incorporated by reference in its entirety.

### BACKGROUND

Mucositis is a frequent and incapacitating complication of intensive chemotherapy and/or radiotherapy. Oral mucositis is the consequence of a direct toxic effect to the oropharyngeal epithelium by chemotherapeutic agents, radiation therapy or a combination of the two approaches in the treatment of cancer. Depending on the localization of the cancer, similar lesions are common in the esophagus secondary to radiation therapy for lung cancer, or in the rectum following prostate cancer therapy, which are specifically referred to as esophagitis and proctitis, respectively. See, for example, S. T. Sonis, Nature Reviews, 2004, 277-284. Disruption of the oral mucosa, in severe cases with ulcers, commonly leads to a debilitating pain that affects eating and often creates a need for opioid analgesics. A severe threat to the patient is that disruption of the biological balance in the oral cavity suddenly gives commensal microorganisms the potential of becoming pathogenic, sometimes culminating in systemic complications including sepsis. In fact, in patients conditioned for hematopoietic stem cell transplantation (HSCT), oral mucositis is associated with an increased risk of dying within 100 days. The increased clinical management cost for affected patients is significant due to more febrile days, lengthened hospital stays and dependence on parenteral nutrition. Searches for patient specific risk factors have been inconclusive, but a link has been established between type and intensity of therapy. While fill recovery can be apparently achieved, the underlying mucosal biology may be permanently changed potentially decreasing the threshold for oral mucositis on relapse therapy.

Oral mucositis is commonly graded using the NCI CTC scale for adverse drug events: Patients with grade 3 and 4 oral mucositis often develop life-threatening complications.

Another complicating factor of oral mucositis is that it often becomes the dose-limiting complication leading to less intense chemo-/radio-therapy possibly reducing cancer survival rate of affected patients. In many cases it must be expected that oral mucositis will be the dose-limiting factor in the development of newer more aggressive cytoreductive therapy combinations that could lead to higher cure rates.

The mucositis lesions are characterized by atrophy of the epithelium and bleeding ulcers and the underlying pathobiology is a complex process that through a series of events, now descriptively divided into four phases, ultimately targets the epithelial stem cells and their capacity to maintain an intact mucosal barrier. Oral mucositis is a complex biology not limited to the epithelium. In the early stages, the inflammatory cell component is limited to resident macrophages. The initial DNA damage and ROS (reactive oxygen species) release leads to activation of several transcription factors: p53, NF-kB, and members of the API transcription factor family, with NF-kB as possibly the most important. The resulting production of pro-inflammatory cytokines (TNFalpha, IL-1) and metalloproteinases, and IL-6 together with a possible shift in pro- and anti-apoptotic signals (BAX and BCL)) stimulates early connective tissue and endothelial damage, and vasodilation, which inturn enhances the delivery of cytotoxic drugs, free radicals and other harmful endogenous mediators to the mucosa, thereby initiating the next phase.

The second phase is characterized by the inhibition of the replication of basal epithelial cells. As the oropharyngeal epithelium is one of the body's tissues with the fastest cell turnover rate, a reduced basal cell activity can no longer keep up with an increased demand further accelerated by toxic epithelial cell death, leading to epithelial cell death and breakdown of the mucosal barrier with ulcer formation. There is also a significant early upregulation of cox-2. Active fibroblasts, through API, release MMPs that add to tissue breakdown. In the third phase of ulcer formation, bacteria are allowed to penetrate the submucosa, further amplifying the proinflammatory response and ulcer formation through the immune response to colonizing bacteria. In a fourth and final phase, spontaneous healing occurs. Normally, oral mucositis rapidly reverses after termination of cancer therapy.

Currently the treatment options for cancer therapy-induced oral mucositis are very limited and most patents receive only supportive therapy. In fact, oral mucositis represents an important complication to control in order to successfully treat patients affected by cancer

### SUMMARY OF INVENTION

The present invention provides methods of treating or preventing mucositis. Such mucositis may be induced by chemotherapy or radiation therapy, and treatment may include improving survival rates by reducing the incidence of therapy-induced mucositis comprising administering a compound of the invention.

### DETAILED DESCRIPTION OF THE DRAWINGS

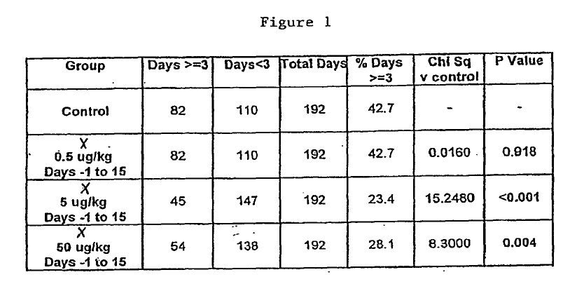

FIG. 1 shows the Chi-squared analysis of days with a score of 3 or higher after administration of compound X versus control.

### DETAILED DESCRIPTION OF THE INVENTION

The present invention provides a method of treating or preventing mucositis comprising administering a compound of formula A, compound of any one of formulae 1 to 46, lipoxin compound, or oxylipin compound, or a combination of aspirin and an omega-3 fatty acid. Mucositis, for the purposes of this application, refers to mucosal injury induced or associated with the administration of radiation or drugs (chemotherapy) for the treatment of cancer and related diseases. Mucositis typically manifests itself as ulcerations, tissue necrosis, and atrophy of the mucous membranes anywhere along the digestive tract, from the mouth to the anus. For example, the present methods may be used to treat ulcerations and tissue necrosis associated with radiation therapy and/or chemotherapy.

The present invention provides a method of preventing the development of chemotherapy or radiation therapy-induced mucositis comprising administering a compound of formula A, compound of any one of formulae 1 to 46, lipoxin compound, or oxylipin compound, or a combination of aspirin and an omega-3 fatty acid. In certain embodiments, a compound of formula A, compound of any one of formulae 1 to 46, lipoxin compound, or oxylipin compound, or a combination of aspirin and an omega-3 fatty acid is administered conjointly with chemotherapy or radiation therapy.

The present invention provides a method of improving survival rates by reducing the incidence of therapy-induced mucositis comprising administering a compound of formula A, compound of any one of formulae 1 to 46, lipoxin compound, or oxylipin compound, or a combination of aspirin and an omega-3 fatty acid. The rate of life-threatening severe mucositis, grade 4 on WHO scale, would be expected to be reduced from an average incidence of 60% in untreated patients, to 20% or less in patents receiving a subject treatment.

Compounds suitable for use in methods of the invention include those of Formula A,

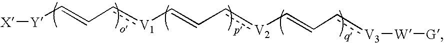

wherein:

each of W′ and Y′ is a bond or a linker independently selected from a ring containing up to 20 atoms or a chain of up to 20 atoms, provided that W′ and Y′ can independently include one or more nitrogen, oxygen, sulfur or phosphorous atoms, further provided that W′ and Y′ can independently include one or more substituents independently selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, or sulfonyl, further provided that W′ and Y′ can independently contain one or more fused carbocyclic, heterocyclic, aryl or heteroaryl rings, and further provided that when o′ is 0, and V₁ is

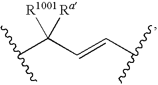

Y′ is connected to V₁ via a carbon atom;

V₁ is selected from

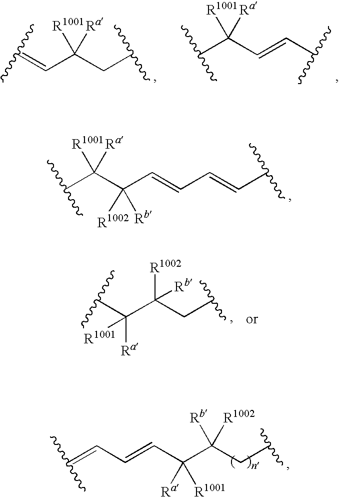

wherein when q′ is 0 and V₃ is a bond, n′ is 0 or 1; otherwise n′ is 1;

V₂ is selected from a bond,

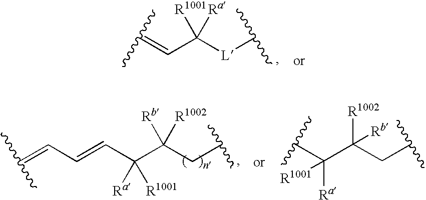

wherein:
L′ is selected from —C(R¹⁰⁰³)(R¹⁰⁰⁴)—, wherein each of R¹⁰⁰³ and R¹⁰⁰⁴ is independently selected from hydrogen, alkyl, alkenyl, alkynyl, perfluoroalkyl, alkoxy, aryl or heteroaryl, or R¹⁰⁰³ and R¹⁰⁰⁴ are connected together to form a carbocyclic or heterocyclic ring; when V₃ is

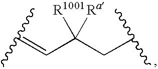

L′ is additionally selected from W′; and n′ is 0 or 1;

V₃ is selected from a bond or

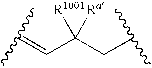

wherein:
each R¹⁰⁰¹ and R¹⁰⁰² is independently for each occurrence selected from hydrogen, alkyl, alkenyl, alkynyl, aryl, heteroaryl, alkylaryl, alkoxy, or halo, wherein said alkyl- or aryl-containing moiety is optionally substituted with up to 3 independently selected substituents;
each of R^{a′} and R^{b′} is independently for each occurrence selected from —OR′ or —N(R′)₂, or adjacent R^{a′} and R^{b′} are taken together to form an epoxide ring having a cis or trans configuration, wherein each R′ is independently selected from hydrogen, alkyl, alkenyl, alkynyl, aryl, heteroaryl, acyl, silyl, alkoxyacyl, aminoacyl, aminocarbonyl, alkoxycarbonyl, or a protecting group;

or when V₁ is

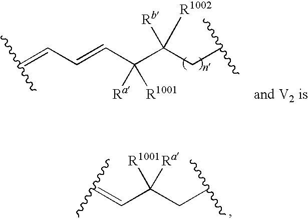

R¹⁰⁰² and R^{b′} are both hydrogen;

X′ is selected from —CN, —C(NH)N(R″)(R″), —C(S)-A′, —C(S)R″, —C(O)-A′, —C(O)—R″, —C(O)—SR″, —C(O)—NH—S(O)₂—R″, —S(O)₂-A′, —S(O)₂—R″, S(O)₂N(R″)(R″), —P(O)₂-A′, —PO(OR″)-A′, -tetrazole, alkyltetrazole, or —CH₂OH, wherein
A′ is selected from —OR″, —N(R″)(R″) or —OM′;
each R″ is independently selected from hydrogen, alkyl, aryl, arylalkyl, heteroaryl, heteroarylalkyl or a detectable label molecule, wherein any alkyl-, aryl- or heteroaryl-containing moiety is optionally substituted with up to 3 independently selected substituents; and
M′ is a cation;

G′ is selected from hydrogen, halo, hydroxy, alkyl, aryl, arylalkyl, heteroaryl, heteroarylalkyl, alkoxy, aryloxy, carboxy, amino, alkylamino, dialkylamino, acylamino, carboxamido or a detectable label molecule, wherein any alkyl-, aryl- or heteroaryl-containing moiety is optionally substituted with up to 3 independently selected substituents;

o′ is 0, 1, 2, 3, 4, or 5;

p′ is 0, 1, 2, 3, 4, or 5;

q′ is 0, 1, or 2; and

o′+p′+q′ is 1, 2, 3, 4, 5 or 6;

wherein:

if V₂ is a bond, then q′ is 0, and V₃ is a bond;

if V₃ is

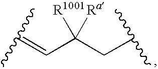

then o′ is 0 V₁ is

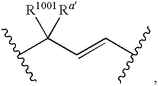

p′ is 1 and V₂ is

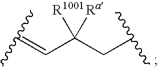

any acyclic double bond may be in a cis or a trans configuration or is optionally replaced by a triple bond; and

either one

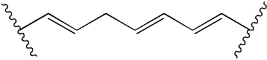

portion of the compound, if present, is optionally replaced by

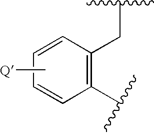

or one

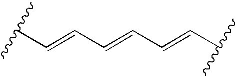

portion of the compound, if present, is optionally replaced by

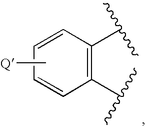

wherein Q′ represents one or more substituents and each Q′ is independently selected from halo, alkyl, alkenyl, alkynyl, cycloalkyl, aryl, heteroaryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl.

In certain embodiments, V₁ is selected from

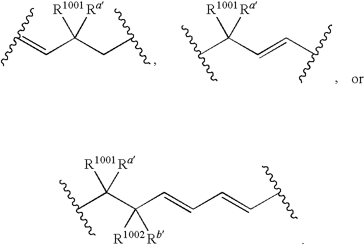

In certain embodiments, V₂ is selected from a bond,

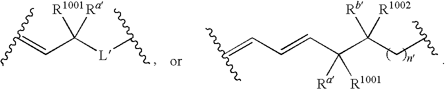

In certain embodiments, when q′ is 0 and V₃ is a bond, n′ is 0 or 1; otherwise n′ is 1.

In certain embodiments, p′ is 0, 1, 2, 3, or 5.

In certain embodiments, q′ is 0 or 1.

In certain embodiments, if V₁ is

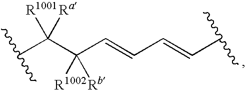

then o′ is 0 or 1, p′ is 1 or 2, o′+p′ is 1 or 2, V₂ is

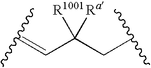

and V₃ is a bond.

In certain embodiments, if V₁ is

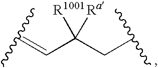

then o′ is 3, 4 or 5, p′ is 0, 1 or 2, o′+p′ is 4 or 5, and V₂ is a bond.

In certain embodiments, if V₂ is a bond, then o′ is 0, 3, 4 or 5; p′ is 0, 1, 2 or 5, o′+p′ is 4 or 5, q′ is 0, and V₃ is a bond.

In certain embodiments, each of W′ and Y′ is independently selected from a bond or lower alkyl or heteroalkyl optionally substituted with one or more substituents independently selected from alkenyl, alkynyl, aryl, chloro, iodo, bromo, fluoro, hydroxy, amino, or oxo.

Compounds suitable for use in methods of the invention include those of Formula 1,

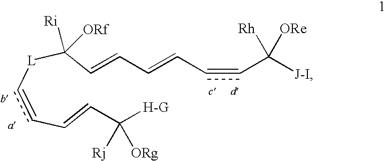

wherein
Carbons a′ and b′ are connected by a double bond or a triple bond;
Carbons c′ and d′ are connected by a double bond or a triple bond;
Re, Rf, and Rg are independently selected from hydrogen, alkyl, alkenyl, alkynyl, aryl, heteroaryl, acyl (e.g., alkoxyacyl, aminoacyl), aminocarbonyl, alkoxycarbonyl, or silyl;
Rh, Ri and Rj are independently selected from hydrogen, alkyl, alkenyl, alkynyl, perfluoroalkyl, aryl or heteroaryl;
I is selected from —C(O)-E, —SO₂-E, —PO(OR)-E, where E is hydroxy, alkoxy, aryloxy, amino, alkylamino, dialkylamino, or arylamino; and R is hydrogen or alkyl;
J, L and H are linkers independently selected from a ring containing up to 20 atoms or a chain of up to 20 atoms, provided that J, L and H can independently include one or more nitrogen, oxygen, sulfur or phosphorous atoms, and further provided that J, L and H can independently include one or more substituents selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that J, L and H can also contain one or more fused carbocyclic, heterocyclic, aryl or heteroaryl rings, and provided that linker J is connected to the adjacent C(R)OR group via a carbon atom;
G is selected from hydrogen, alkyl, perfluoroalkyl, alkenyl, alkynyl, aryl, heteroaryl, chloro, iodo, bromo, fluoro, hydroxy, alkoxy, aryloxy, carboxy, amino, alkylamino, dialkylamino, acylamino, or carboxamido;
or pharmaceutically acceptable salts thereof.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

In certain embodiments, a compound of formula 1 is represented by formula 2,

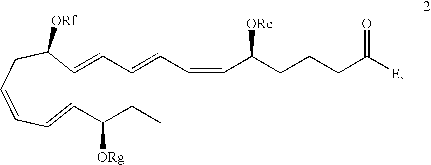

wherein
E, Re, Rf, and Rg are as defined above.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Exemplary compounds of formula 2 include:

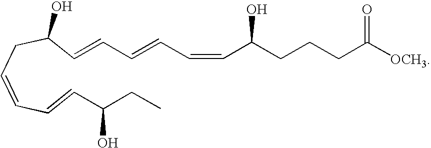

In certain embodiments, a compound of formula 1 is represented by formula 3,

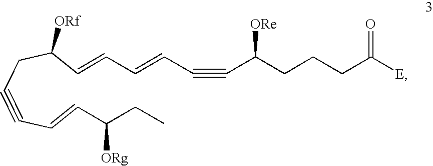

wherein
E, Re, Rf, and Rg are as defined above.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Exemplary compounds of formula 3 include:

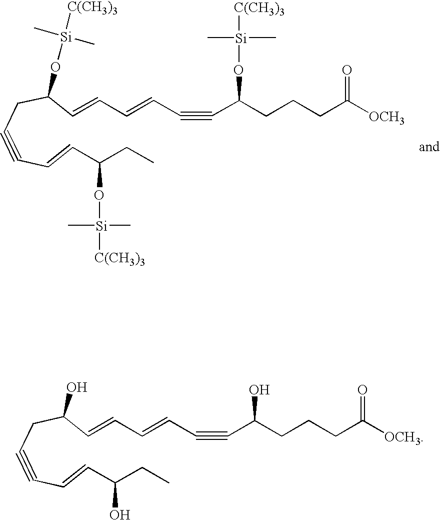

Other compounds suitable for use in methods of the invention include those of Formula 4,

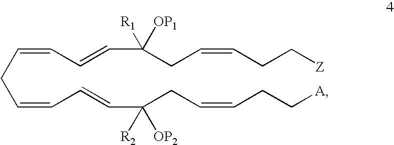

wherein
A is H or —OP₄;
P₁, P₂ and P₄ each individually is a protecting group or hydrogen atom;
R₁ and R₂ each individually is a substituted or unsubstituted, branched or unbranched alkyl, alkenyl, or alkynyl group, substituted or unsubstituted aryl group, substituted or unsubstituted, branched or unbranched alkylaryl group, halogen atom, hydrogen atom;
Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN, preferably a carboxylic acid, ester, amide, thioester, thiocarboxamide or a nitrile;
each R^{a}, if present, is independently selected from hydrogen, (C1-C6) alkyl, (C2-C6) alkenyl, (C2-C6) alkynyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered heterocyclyl, morpholinyl, piperazinyl, homopiperazinyl, piperidinyl, 4-11 membered heterocyclylalkyl, 5-10 membered heteroaryl and 6-16 membered heteroarylalkyl;
each R^{b}, if present, is a suitable group independently selected from ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR′, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, [NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};
each R^{c}, if present, is independently a protecting group or R^{a}, or, alternatively, two R^{c} taken together with the nitrogen atom to they are bonded form a 5 to 8-membered heterocyclyl or heteroaryl which optionally including one or more additional heteroatoms and optionally substituted with one or more of the same or different R^{a} or suitable R^{b} groups;
each n independently is an integer from 0 to 3;
each R^{d} independently is a protecting group or R^{a};
or pharmaceutically acceptable salts thereof.

Other compounds suitable for use in methods of the invention include those of Formula 5,

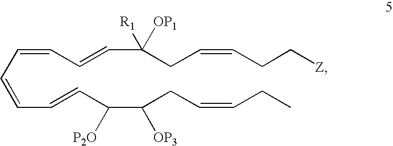

or pharmaceutically acceptable salts thereof, wherein
P₃ is a protecting group or hydrogen atom; and
P₁, P₂, R₁ and Z are as defined above in formula 4.

Other compounds suitable for use in methods of the invention include those of Formula 6,

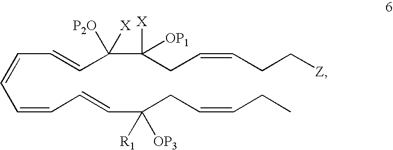

or pharmaceutically acceptable salts thereof, wherein
each X represents hydrogen or taken together both X groups represent one substituted or unsubstituted methylene, an oxygen atom, a substituted or unsubstituted N atom, or a sulfur atom such that a three-membered ring is formed; and
P₁, P₂, P₃, R₁ and Z are as defined above.

Other compounds suitable for use in methods of the invention include those of Formula 7,

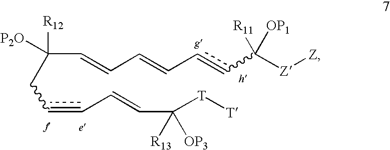

or pharmaceutically acceptable salts thereof, wherein
Carbons e′ and f′ are connected by a double bond or a triple bond, and when carbon e′ is connected to carbon f′ through a double bond the stereochemistry is cis or trans;
Carbons g′ and h′ are connected by a double bond or a triple bond and when carbon g′ is connected to carbon h′ through a double bond the stereochemistry is cis or trans;
m is 0 or 1;
T′ is hydrogen, (C1-C6) alkyl, (C2-C6) alkenyl, (C2-C6) alkynyl, (C5-C14) aryl, (C6-C16) arylalkyl, 5-14 membered heteroaryl, 6-16 membered heteroarylalkyl, or —CH═CHCH₂CH₃;
T is —(CH₂)_{q}— or —(CH₂)_{q}—O—, where q is an integer from 0 to 6;
Z′ is (C1-C6) alkylene optionally substituted with 1, 2, 3, 4, 5 or 6 of the same or different halogen atoms, —(CH₂)ₚ—O—CH₂— or —(CH₂)ₘ—S—CH₂—, where p is an integer from 0 to 4;
R₁₁, R₁₂ and R₁₃ each individually is substituted or unsubstituted, branched or unbranched alkyl, alkenyl, or alkynyl group, substituted or unsubstituted aryl group, substituted or unsubstituted, branched or unbranched alkylaryl group, C₁₋₄alkoxy, halogen atom, —CH₂R₁₄, —CHR₁₄R₁₄, —CR₁₄R₁₄R₁₄, or a hydrogen atom;
   R₁₄ is independently for each occurrence selected from —CN, —NO₂ or halogen;
   P₁, P₂, P₃; and Z are as defined above.

Other compounds suitable for use in methods of the invention include those of Formula 8,

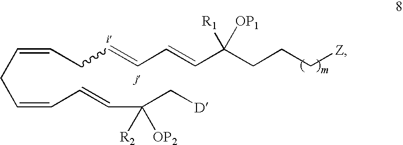

or pharmaceutically acceptable salts thereof, wherein
the stereochemistry of the carbon i′ to carbon j′ bond is cis or trans;
m is 0 or 1;
D′ is CH₃, —CH═CHCH₂U or —CH═CHCH₂CH₂A;
U is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;
A is H or —OP₄;
P₁, P₂, P₄, R₁, R₂ and Z are as defined above.

Other compounds suitable for use in methods of the invention include those of Formula 9,

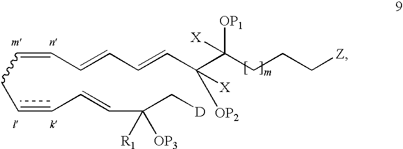

or pharmaceutically acceptable salts thereof, wherein
Carbons k′ and l′ are connected by a double bond or a triple bond;
the stereochemistry of the carbon m′ to carbon n′ double bond is cis or trans;
m is 0 or 1;
D is —CH₃ or —CH═CHCH₂CH₃;
P₁, P₂, P₃, R₁, X, and Z are as defined above.

Other compounds suitable for use in methods of the invention include those of Formula 10,

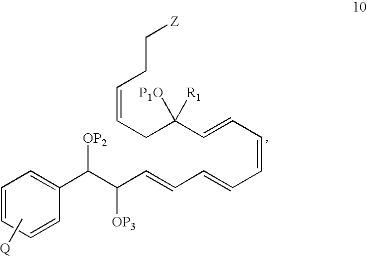

or pharmaceutically acceptable salts thereof, wherein
P₁, P₂, P₃, R₁ and Z are as defined above; and
Q represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group.

Other compounds suitable for use in methods of the invention include those of Formula 11,

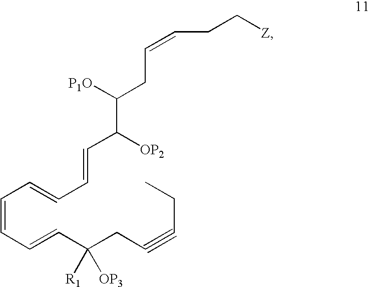

or pharmaceutically acceptable salts thereof, wherein
P₁, P₂, P₃, R₁, and Z are as defined above.

Other compounds suitable for use in methods of the invention include those of Formula 12,

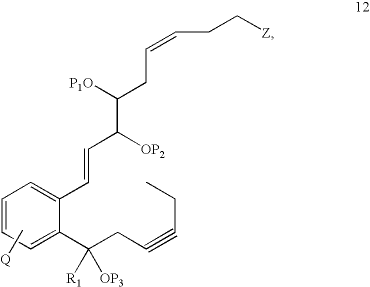

or pharmaceutically acceptable salts thereof, wherein
P₁, P₂, P₃, Q, R₁, and Z are as defined above.

Other compounds suitable for use in methods of the invention include those of Formula 13,

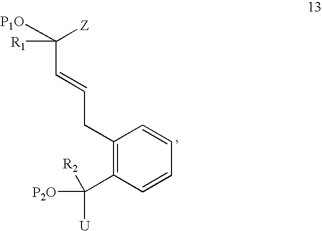

or pharmaceutically acceptable salts thereof, wherein
P₁, P₂, R₁, R₂, U, and Z are as defined above.

Other compounds suitable for use in methods of the invention include those of Formula 14,

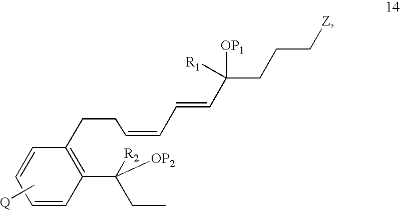

or pharmaceutically acceptable salts thereof, wherein
P₁, P₂, R₁, R₂, Q, and Z are as defined above.

Other compounds suitable for use in methods of the invention include those of Formula 15,

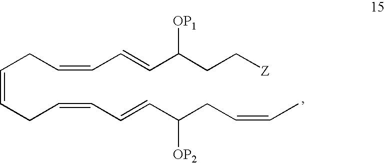

or pharmaceutically acceptable salts thereof, wherein
P₁, P₂, and Z are as defined above.

Other compounds suitable for use in methods of the invention include those of Formula 16,

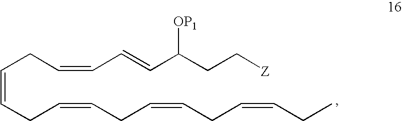

or pharmaceutically acceptable salts thereof, wherein
P₁ and Z are as defined above.

Other compounds suitable for use in methods of the invention include those of Formula 17,

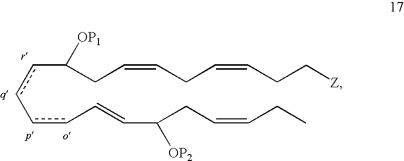

or pharmaceutically acceptable salts thereof, wherein
Carbons o′ and p′ are connected by a single or a double bond;
Carbons q′ and r′ are connected by a single or a double bond; and
P₁, P₂, and Z are as defined above

Other compounds suitable for use in methods of the invention include those of Formula 18,

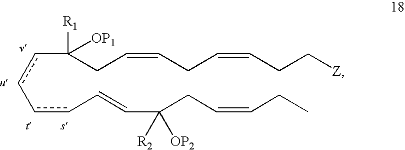

or pharmaceutically acceptable salts thereof, wherein
the stereochemistry of the carbon s′ to carbon t′ double bond is cis or trans;
the stereochemistry of the carbon u′ to carbon v′ double bond is cis or trans; and
P₁, P₂, R₁, R₂, and Z are as defined above

Other compounds suitable for use in methods of the invention include those of Formula 19,

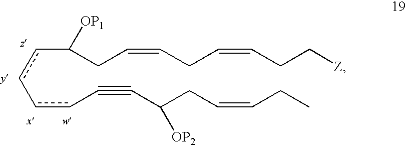

or pharmaceutically acceptable salts thereof, wherein
Carbons w′ and x′ are connected by a single or a double bond;
Carbons y′ and z′ are connected by a single or a double bond; and
P₁, P₂, and Z are as defined above.

In certain embodiments of formulae 4 to 19, each R^{b}, if present, is a suitable group independently selected from ═O, —OR , (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, [NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c}.

Other compounds suitable for use in methods of the invention include those of Formula 20,

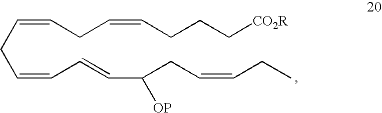

Formula 21,

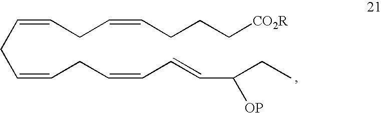

Formula 22,

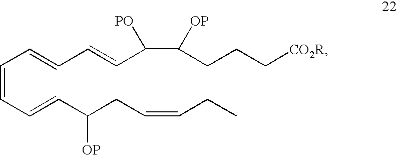

Formula 23,

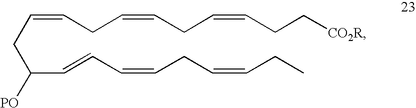

Formula 24,

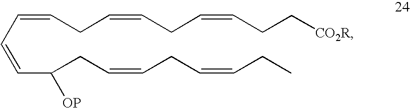

Formula 25,

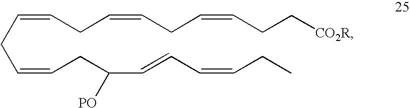

Formula 26,

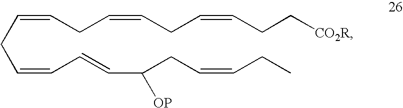

Formula 27,

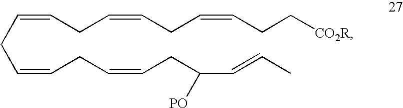

or Formula 28,

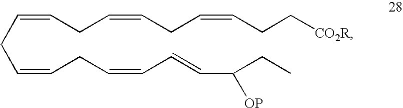

or pharmaceutically acceptable salts of any of the above, wherein
each P is individually selected from H or a protecting group; and
R is H, C₁₋₆alkyl (e.g., methyl, ethyl, glycerol), C₂₋₆alkenyl or C₂₋₆alkynyl.

Other compounds suitable for use in methods of the invention include those of Formula 29,

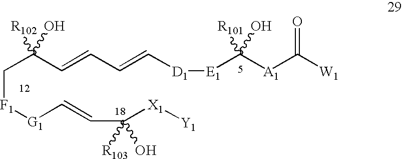

and pharmaceutically acceptable salts, hydrates and solvates thereof, wherein:
D₁-E₁ and F₁-G₁ are independently are cis or trans —C═C— or —C≡C—;
R₁₀₁, R₁₀₂ and R₁₀₃ are independently selected from hydrogen, (C1-C4) straight-chained or branched alkyl, (C2-C4) alkenyl, (C2-C4) alkynyl, (C1-C4) alkoxy, —CH₂R₁₀₄, —CHR₁₀₄R₁₀₄ and —CR₁₀₄R₁₀₄R₁₀₄;
each R₁₀₄ is independently selected from CN, —NO₂ and halogen;
W₁ is selected from —R₁₀₅, —OR₁₀₅, —SR₁₀₅ and —NR₁₀₅R₁₀₅;
each R₁₀₅ is independently selected from hydrogen, (C1-C6) alkyl, (C2-C6) alkenyl or (C2-C6) alkynyl optionally substituted with one or more of the same or different R groups, (C5-C14) aryl optionally substituted with one or more of the same or different R groups, phenyl optionally substituted with one or more of the same or different R groups, (C6-C16) arylalkyl optionally substituted with one or more of the same or different R groups, 5-14 membered heteroaryl optionally substituted with one or more of the same or different R groups, 6-16 membered heteroarylalkyl optionally substituted with one or more of the same or different R groups and a detectable label molecule;
A₁ is selected from (C1-C6) alkylene optionally substituted with 1, 2, 3, 4, 5 or 6 of the same or different halogen atoms, —(CH₂)ₘ—O—CH₂— and —(CH₂)ₘ—S—CH₂—, where m is an integer from 0 to 4;
X₁ is selected from —(CH₂)ₙ— and —(CH₂)ₙ—O—, where n is an integer from 0 to 6;
Y₁ is selected from hydrogen, (C1-C6) alkyl, (C2-C6) alkenyl, or (C2-C6) alkynyl, optionally substituted with one or more of the same or different R₁₀₀ groups, (C5-C14) aryl optionally substituted with one or more of the same or different R₁₀₀ groups, phenyl, optionally substituted with one or more of the same or different R₁₀₀ groups, (C6-C16) arylalkyl optionally substituted with one or more of the same or different R₁₀₀ groups, 5-14 membered heteroaryl optionally substituted with one or more of the same or different R₁₀₀ groups, 6-16 membered heteroarylalkyl optionally substituted with one or more of the same or different R₁₀₀ groups and a detectable label molecule;
each R₁₀₀ is independently selected from an electronegative group, ═O, —OR^{a1}, (C1-C3) haloalkyloxy, ═S, —SR^{a1}, ═NR^{a1}, ═NONR^{a1}, —NR^{c1}R^{c1}, halogen, —CF₃, —CN, —NC, —OCN, —SCN, —NO, —NO₂, ═N₂, —N₃, —S(O)R^{a1}, —S(O)₂R^{a1}, —S(O)₂OR^{a1}, —S(O)₂NR^{c1}R^{c1}, —OS(O)R^{a1}, —OS(O)₂R^{a1}, —OS(O)₂OR^{a1}, —OS(O)₂NR^{c1}R^{c1}, —C(O)R^{a1}, —C(O)OR^{a1}, —C(O)NR^{c1}R^{c1}, —C(NH)NR^{c1}R^{c1}, —OC(O)R^{a1}, —OC(O)OR^{a1}, —OC(O)NR^{c1}R^{c1}, —OC(NH)NR^{c1}R^{c1}, —NHC(O)R^{a1}, —NHC(O)OR^{a1}, —NHC(O)NR^{c1}R^{c1} and —NHC(NH)NR^{c1}R^{c1};
each R^{a1} is independently selected from hydrogen, (C1-C4) alkyl, (C2-C4) alkenyl or (C2-C4) alkynyl; and
   each R^{c1} is independently an R^{a1} or, alternatively, R^{c1}R^{c1} taken together with the nitrogen atom to which it is bonded forms a 5 or 6 membered ring.

In certain embodiments of Formula 29, when X₁—Y₁ is —CH₂CH₃, then at least one of R₁₀₁, R₁₀₂ or R₁₀₃ is other than hydrogen.

In certain embodiments, a compound of Formula 29 is represented by Formula 30,

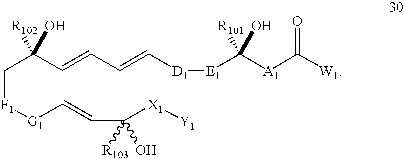

Other compounds suitable for use in methods of the invention include those of Formulae 31 to 37

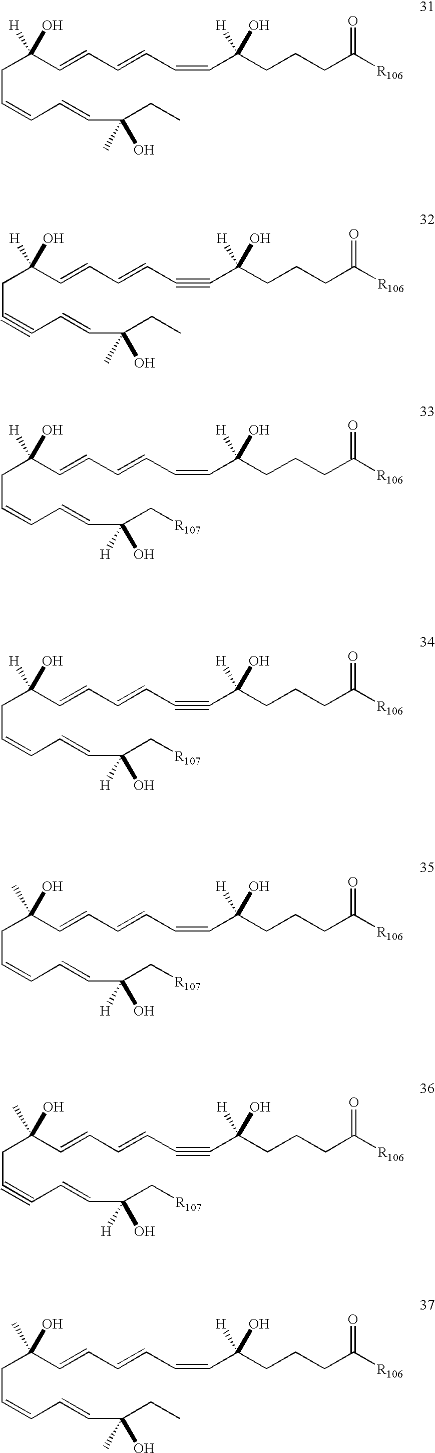

and pharmaceutically acceptable salts, hydrates and solvates thereof,

wherein
R₁₀₆ is —OH, —OCH₃, —OCH(CH₃)₂ or —NHCH₂CH₃; and

### R₁₀₇ is

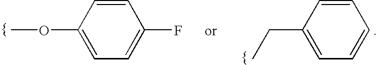

Other compounds suitable for use in methods of the invention include those of Formula 38,

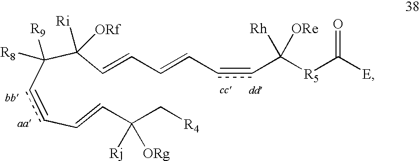

wherein
Carbons aa′ and bb′ are connected by a double bond or a triple bond;
Carbons cc′ and dd′ are connected by a double bond or a triple bond;
Re, Rf, and Rg are independently selected from hydrogen, alkyl, alkenyl, alkynyl, aryl, heteroaryl, acyl (e.g., alkoxyacyl, aminoacyl), aminocarbonyl, alkoxycarbonyl, or silyl;
E is hydroxyl, alkoxy, aryloxy, amino, alkylamino, dialkylamino, or arylamino;
Rh, Ri and Rj are independently selected from hydrogen, alkyl, alkenyl, alkynyl, perfluoroalkyl, aryl or heteroaryl;
R₄ is selected from hydrogen, alkyl, perfluoroalkyl, alkenyl, alkynyl, aryl, heteroaryl, fluoro, hydroxyl, alkoxy, aryloxy;
R₅ is selected from i-iv as follows: i) CH₂CH(R₆)CH₂, where R₆ is hydrogen, alkyl, alkenyl, alkynyl, perfluoroalkyl, aryl, heteroaryl, fluoro, hydroxyl or alkoxy; ii) CH₂C(R₆R₇)CH₂, where R₆ and R₇ are each independently alkyl, alkenyl, alkynyl, perfluoroalkyl, aryl, or fluoro, or R₆ and R₇ are connected together to form a carbocyclic or heterocyclic ring; iii) CH₂OCH₂, CH₂C(O)CH₂, or CH₂CH₂; or iv) R₅ is a carbocyclic, heterocyclic, aryl or heteroaryl ring; and
R₈ and R₉ are independently selected from hydrogen, alkyl, alkenyl, alkynyl, perfluoroalkyl, alkoxy, aryl or heteroaryl, or R₈ and R₉ are connected together to form a carbocyclic or heterocyclic ring;
   or pharmaceutically acceptable salts thereof.

In certain embodiments R₈ and R₉ are hydrogen.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Other compounds suitable for use in methods of the invention include those of Formulae 39-44,

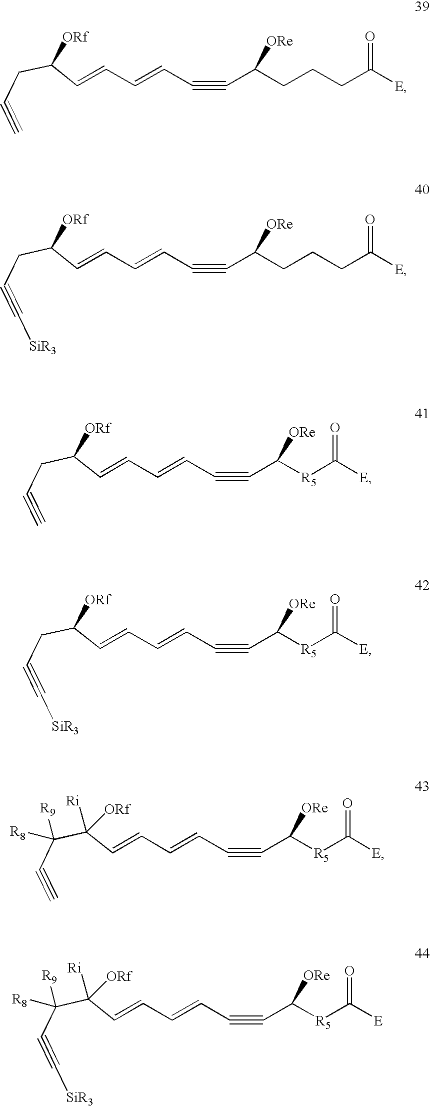

and pharmaceutically acceptable salts thereof, wherein
Re, Rf, E, Ri, R₅, R₈ and R₉ are as defined above.

Exemplary compounds of formulae 39, 41, and 43 include:

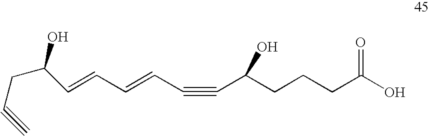

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Other compounds suitable for use in methods of the invention include those of Formula 46,

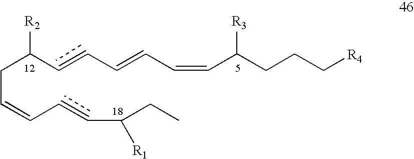

or a pharmaceutically acceptable salt or prodrug thereof, wherein:
each [symbol] independently designates a double or triple bond;
R₁, R², and R³ are each independently OR, OX¹, SR, SX², N(R)₂, NHX³, NRC(O)R, NRC(O)N(R)₂, C(O)OR, C(O)N(R)₂, SO₂R, NRSO₂R, C(O)R, or SO₂N(R)₂;
each R is independently selected from hydrogen or an optionally substituted group selected from C₁₋₆ aliphatic, a 3-8 membered saturated, partially unsaturated, or aryl ring having 0-4 heteroatoms independently selected from nitrogen, oxygen, or sulfur, or;
two R on the same nitrogen are taken together with the nitrogen to form a 5-8 membered heterocyclyl or heteroaryl ring having 1-3 heteroatoms independently selected from nitrogen, oxygen, or sulfur;
each X¹ is independently a suitable hydroxyl protecting group;
each X² is independently a suitable thiol protecting group;
each X³ is independently a suitable amino protecting group; and
R⁴ is NRC(O)R, NRC(O)N(R)₂, C(O)OR, C(O)N(R)₂, SO₂R, NRSO₂R, C(O)R, or SO₂N(R)₂.

The compounds above (e.g., compounds of formula A or formulae 1 to 46) are known to be useful in the treatment or prevention of inflammation or inflammatory disease. Examples of such compounds are disclosed in the following patents and applications: US 2003/0191184, WO 2004/014835, WO 2004/078143, U.S. Pat. No. 6,670,396, US 2003/0236423, US 2005/0228047, US 2005/0238589 and US2005/0261255. These compounds are suitable for use in methods of the present invention.

Other compounds useful in this invention are compounds that are chemically similar variants to any of the compounds of formula A or formulae 1 to 46 set forth above. The term “chemically similar variants” includes, but is not limited to, replacement of various moieties with known biosteres; replacement of the end groups of one of the compounds above with a corresponding end group of any other compound above, modification of the orientation of any double bond in a compound, the replacement of any double bond with a triple bond in any compound, and the replacement of one or more substituents present in one of the compounds above with a corresponding substituent of any other compound.

Lipoxin compounds suitable for use in this invention include those of formula 50:

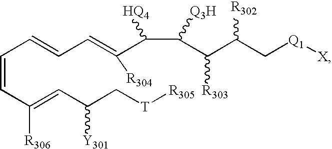

wherein:

X is R₃₀₁, OR₃₀₁, or SR₃₀₁;

R₃₀₁ is
(a) a hydrogen atom;
(b) an alkyl of 1 to 8 carbons atoms, inclusive, which may be straight chain or branched;
(c) a cycloalkyl of 3 to 10 carbon atoms;
(d) an aralkyl of 7 to 12 carbon atoms;
(e) phenyl;
(f) substituted phenyl

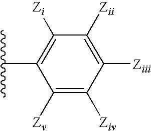

wherein Zᵢ Zᵢᵢ, Zᵢᵢᵢ, Zᵢᵥ and Zᵥ are each independently selected from —NO₂, —CN, —C(═O)—R₃₀₁, —SO₃H, a hydrogen atom, halogen, methyl, —ORₓ, wherein Rx is 1 to 8 carbon atoms, inclusive, which may be a straight chain or branched, and hydroxyl, wherein when any of Zᵢ Zᵢᵢ, Zᵢᵢᵢ, Zᵢᵥ or Zᵥ is C(═O)—R₃₀₁, said Zᵢ Zᵢᵢ, Zᵢᵢᵢ, Zᵢᵥ or Zᵥ is not substituted with another C(═O)—R₃₀₁.
(g) a detectable label molecule; or
(h) a straight or branched chain alkenyl of 2 to 8 carbon atoms, inclusive;

Q₁ is (C═O), SO₂ or (CN), provided when Q₁ is CN, then X is absent;

Q₃ and Q₄ are each independently O, S or NH;

one of R₃₀₂ and R₃₀₃ is a hydrogen atom and the other is:
(a) H;
(b) an alkyl of 1 to 8 carbon atoms, inclusive, which may be a straight chain or branched;
(c) a cycloalkyl of 3 to 6 carbon atoms, inclusive;
(d) an alkenyl of 2 to 8 carbon atoms, inclusive, which may be straight chain or branched; or
(e) RₖQ₂R₁ wherein Q₂ is —O— or —S—; wherein Rₖ is alkylene of 0 to 6 carbons atoms, inclusive, which may be straight chain or branched and wherein R₁ is alkyl of 0 to 8 carbon atoms, inclusive, which may be straight chain or branched, provided when R₁ is 0, then R₁ is a hydrogen atom;

R₃₀₄ is
(a) H;
(b) an alkyl of 1 to 6 carbon atoms, inclusive, which may be a straight chain or branched;

R₃₀₅ is

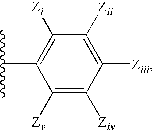

wherein Zᵢ Zᵢᵢ, Zᵢᵢ, Zᵢᵥ and Zᵥ are defined as above;

R₃₀₆ is
(a) H;
(b) an alkyl from 1 to 4 carbon atoms, inclusive, straight chain or branched;

wherein Y₃₀₁ is —OH, methyl, —SH, an alkyl of 2 to 4 carbon atoms, inclusive, straight chain or branched, an alkoxy of 1 to 4 carbon atoms, inclusive, or (CH)ₚ(Z)_{q}, where p+q=3, p=0 to 3, q=0 to 3 and Z is cyano, nitro or a halogen; and

T is O or S;

and pharmaceutically acceptable salts thereof.

Lipoxin compounds suitable for use in this invention include those of formulae 51, 52, 53 or 54:

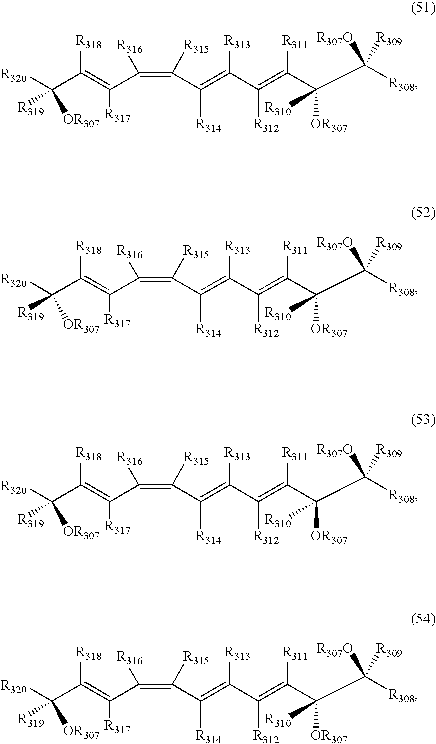

wherein:

each R₃₀₇ is independently selected from hydrogen and straight, branched, cyclic, saturated, or unsaturated alkyl having from 1 to 20 carbon atoms;

R₃₀₈, R₃₀₉, R₃₁₀, R₃₁₉, and R₃₂₀ are independently selected from:
(a) hydrogen;
(b) straight, branched, cyclic, saturated, or unsaturated alkyl having from 1 to 20 carbon atoms;
(c) substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl;
(d) substituted aryl or heteroaryl, wherein the aryl or heteroaryl is substituted with one or more substituents selected from alkyl, cycloalkyl, alkoxy, halo, aryl, heteroaryl, carboxyl, and carboxamido; and
(e) Z—Y, wherein:

Z is selected from a straight, branched, cyclic, saturated, or unsaturated alkyl having from 1 to 20 carbon atoms; substituted lower alkyl, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl; and substituted aryl or heteroaryl, wherein the aryl or heteroaryl is substituted with one or more substituents selected from alkyl, cycloalkyl, alkoxy, halo, aryl, heteroaryl, carboxyl, and carboxamido; and

Y is selected from hydrogen; alkyl; cycloalkyl; carboxyl; carboxamido; aryl; heteroaryl; substituted aryl or heteroaryl, wherein the aryl or heteroaryl is substituted with one or more substituents selected from alkyl, cycloalkyl, alkoxy, halo, aryl, heteroaryl, carboxyl, and carboxamido; and

R₃₁₁ to R₃₁₈ are independently selected from:
(a) hydrogen;
(b) halo;
(c) straight, branched, cyclic, saturated, or unsaturated alkyl having from 1 to 20 carbon atoms;
(d) substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl;
(e) substituted aryl or heteroaryl, wherein the aryl or heteroaryl is substituted with one or more substituents selected from alkyl, cycloalkyl, alkoxy, halo, aryl, heteroaryl, carboxyl, and carboxamido; or

R₃₀₈ to R₃₂₀ are independently a bond that forms a carbon-carbon double bond, a carbon-carbon triple bond, or a ring with the lipoxin backbone; or

any two of R₃₀₇ to R₃₂₀ are taken together with the atoms to which they are bound and optionally to 1 to 6 oxygen atoms, 1 to 6 nitrogen atoms, or both 1 to 6 oxygen atoms and 1 to 6 nitrogen atoms, to form a ring containing 3 to 20 atoms.

Lipoxin compounds suitable for use in this invention include those of formula 55:

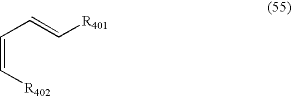

wherein:

R₄₀₁ is selected from:

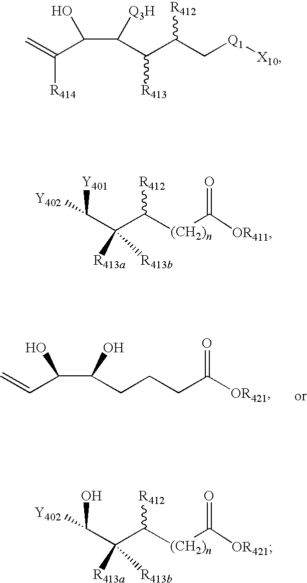

R₄₀₂ is selected from:

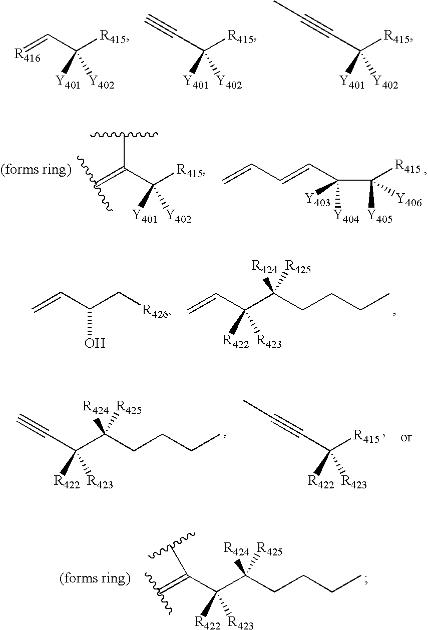

X₁₀ is R₄₁₁, OR₄₁₁, or SR₄₁₁;

R₄₁₁ is
(a) a hydrogen atom;
(b) an alkyl of 1 to 8 carbons atoms, inclusive, which may be straight chain or branched;
(c) a cycloalkyl of 3 to 10 carbon atoms;
(d) an aralkyl of 7 to 12 carbon atoms;
(e) phenyl;
(f) substituted phenyl

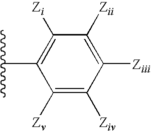

wherein Zᵢ Zᵢᵢ, Zᵢᵢᵢ, Zᵢᵥ and Zᵥ are each independently selected from —NO₂, —CN, —C(═O)—R₄₁₁, —SO₃H, a hydrogen atom, halogen, methyl, —ORₓ, wherein Rₓ is 1 to 8 carbon atoms, inclusive, which may be a straight chain or branched, and hydroxyl; wherein when any of Zᵢ Zᵢ, Zᵢᵢᵢ, Zᵢᵥ or Zᵥ is C(═O)—R₄₁₁, said Zᵢ Zᵢᵢ, Zᵢᵢᵢ, Zᵢᵥ or Zᵥ is not substituted with another C(═O)—R₄₁₁.
(g) a detectable label molecule; or
(h) a straight or branched chain alkenyl of 2 to 8 carbon atoms, inclusive;
Q₁ is (C═O), SO₂ or (CN);
Q₃ is O, S or NH;
one of R₄₁₂ and R₄₁₃ is a hydrogen atom and the other is selected from:
   (a) H;
   (b) an alkyl of 1 to 8 carbon atoms, inclusive, which can be straight chain or branched;
   (c) a cycloalkyl of 3 to 6 carbon atoms, inclusive;
   (d) an alkenyl of 2 to 8 carbon atoms, inclusive, which can be straight chain or branched; or
   (e) R₄₃₁Q₂R₄₃₂ wherein Q₂ is —O— or —S—; wherein R₄₃₁ is alkylene of 0 to 6 carbons atoms, inclusive, which can be straight chain or branched and wherein R₄₃₁ is alkyl of 0 to 8 carbon atoms, inclusive, which can be straight chain or branched;
R₄₁₃ₐ and R_{413b} are each independently:
   (a) H;
   (b) an alkyl of 1 to 8 carbon atoms, inclusive, which can be straight chain or branched;
   (c) a cycloalkyl of 3 to 6 carbon atoms, inclusive;
   (d) an alkenyl of 2 to 8 carbon atoms, inclusive, which can be straight chain or branched; or
   (e) R₄₃₁Q₂R₄₃₂ wherein R₄₃₁, Q₂, and R₄₃₂ are as defined above;
R₄₁₄ is
   (a) H;
   (b) an alkyl of 1 to 6 carbon atoms, inclusive, can be straight chain or branched;
R₄₁₅ is
   (a) an alkyl of 1 to 9 carbon atoms which can be straight chain or branched;
   (b) —(CH₂)—Rᵢ
wherein n=0 to 4 and Rᵢ is
(i) a cycloalkyl of 3 to 10 carbon atoms, inclusive;
   (ii) a phenyl; or
   (iii) substituted phenyl

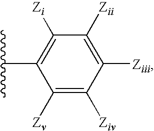

wherein Zᵢ through Zᵥ are as defined above;
(b) R₄₃₁Q₂R₄₃₂, wherein R₄₃₁, Q₂, and R₄₃₂ are as defined above;
(c) —C(Rᵢᵢᵢ)(Rᵢᵥ)—Rᵢ,
wherein Rᵢᵢᵢ and Rᵢᵥ are each independently:
   (i) a hydrogen atom;
   (ii) (CH)ₚ(Z)_{q}, wherein Z, p, and q are as defined above;
(e) a haloalkyl of 1 to 8 carbon atoms, inclusive, and 1 to 6 halogen atoms, inclusive, straight chain or branched;
R₄₁₆ is
   (a) H;
   (b) an alkyl from 1 to 4 carbon atoms, inclusive, straight chain or branched;
   (c) a halogen;
one of Y₄₀₁ or Y₄₀₂ is —OH, methyl, or —SH, and wherein the other is selected from:
   (a) H;
   (b) (CH)ₚ(Z)_{q} where p+q=3, p=0 to 3, q=0 to 3 and each Z, independently, is cyano, nitro or a halogen;
   (c) an alkyl of 2 to 4 carbon atoms, inclusive, straight chain or branched; or
   (d) an alkoxy of 1 to 4 carbon atoms, inclusive,
or Y₄₀₁ and Y₄₀₂ taken together are:
   (d) ═NH; or
   (e) ═O;
one of Y₄₀₃ or Y₄₀₄ is —OH, methyl, or —SH, and wherein the other is selected from:
   (a) H;
   (b) (CH)ₚ(Z)_{q} wherein Z, p, and q are as defined above;
   (c) an alkyl of 2 to 4 carbon atoms, inclusive, straight chain or branched; or
   (d) an alkoxy of 1 to 4 carbon atoms, inclusive,
or Y₄₀₁ and Y₄₀₂ taken together are:
   (a) ═NH; or
   (b) ═O;
one of Y₄₀₅ or Y₄₀₆ is —OH, methyl, or —SH, and wherein the other is selected from:
   (a) H
   (b) (CH)ₚ(Z)_{q} wherein Z, p, and q are as defined above;
   (c) an alkyl of 2 to 4 carbon atoms, inclusive, straight chain or branched; or
   (d) an alkoxy of 1 to 4 carbon atoms, inclusive,
or Y₄₀₁ and Y₄₀₂ taken together are:

1 (a) ═NH; or
(b) ═O;
R₄₂₁ is
   (a) H; or
   (b) alkyl of 1 to 8 carbon atoms;
R₄₂₂ and R₄₂₃ are each independently:
   (a) H;
   (b) a hydroxyl, or a thiol;
   (c) a methyl or a halomethyl;
   (d) a halogen; or
   (e) an alkoxy of 1 to 3 carbon atoms;
R₄₂₄ and R₄₂₅ are each independently:
   (a) H;
   (b) a hydroxyl, or a thiol;
   (c) a methyl or a halomethyl;
   (d) a halogen;
   (e) an alkoxy of 1 to 3 carbon atoms; or
   (f) an alkyl or haloalkyl of 2 to 4 carbon atoms inclusive, which can be straight chain or branched; and
R₄₂₆ is
   (a) a substituted phenyl

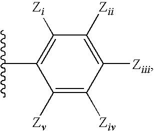

wherein Zᵢ through Zᵥ are as defined above;
(b) a substituted phenoxy

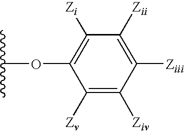

wherein Zᵢ through Zᵥ are as defined above; or
(c)

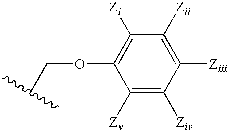

wherein Zᵢ through Zᵥ are as defined above.

Lipoxin compounds suitable for use in this invention include those of formula 56:

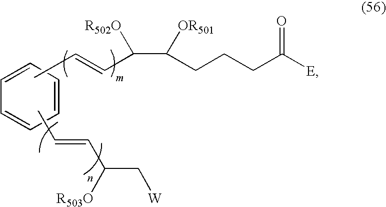

wherein:

E is hydroxy, alkoxy, aryloxy, amino, alkylamino, dialkylamino or —OM, where M is a cation selected from ammonium, tetra-alkyl ammonium, and the cations of sodium, potassium, magnesium and zinc;

W is hydrogen, alkyl, alkenyl, alkynyl, aryl, heteroaryl, halo, hydroxy, alkoxy, aryloxy, carboxy, amino, alkylamino, dialkylamino, acylamino, carboxamido, or sulfonamide;

each of R₅₀₁-R₅₀₃ are independently selected from hydrogen, alkyl, aryl, acyl or alkoxyacyl;

n is 0, 1 or 2;

m is 1 or 2; and

the two substituents on the phenyl ring are ortho, meta, or para.

Lipoxin compounds suitable for use in this invention include those of formula 57:

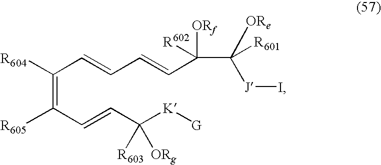

wherein:

I is selected from: —C(O)-E, —SO₂-E, —PO(OR)-E, where E is hydroxy, alkoxy, aryloxy, amino, alkylamino, dialkylamino, or —OM, where M is a cation selected from ammonium, tetra-alkyl ammonium, Na, K, Mg, and Zn; and R is hydroxyl or alkoxy

J′ and K′ are linkers independently selected from a chain of up to 20 atoms and a ring containing up to 20 atoms, provided that J′ and K′ can independently include one or more nitrogen, oxygen, sulfur or phosphorous atoms, and further provided that J′ and K′ can independently include one or more substituents selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that J′ and K′ can also contain one or more fused carbocyclic, heterocyclic, aryl or heteroaryl rings, and provided that linkers J′ and K′ are connected to the adjacent C(R)OR group via a carbon atom or a C-heteroatom bond where the heteroatom is oxygen, sulfur, phosphorous or nitrogen;

G is selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, and carboxamido.

Re, Rf and Rg, are independently selected from hydrogen, alkyl, aryl, heteroaryl, acyl, silyl, alkoxyacyl and aminoacyl;

R₆₀₁, R₆₀₂ and R₆₀₃ are independently selected from hydrogen, alkyl, aryl and heteroaryl, provided that R₆₀₁, R₆₀₂ and R₆₀₃ can independently be connected to linkers J′ or K′;

R₆₀₄ and R₆₀₅ are independently selected from hydrogen, alkyl, alkenyl, alkynyl, aryl, heteroaryl, fluoro, and provided that R₆₀₄ and R₆₀₅ can be joined together to form a carbocyclic, heterocyclic or aromatic ring, and further provided that R₆₀₄ and R₆₀₅ can be replaced by a bond to form a triple bond.

Other compounds suitable for use in methods of the invention are the Oxylipins described in international application WO 2006055965, the compounds in which are incorporated herein by reference. Examples of such compounds are those of formulae 58 to 115, as shown in Table 1. These compounds include long chain omega-6 fatty acids, docosapentaenoic acid (DPAn-6) (compounds 58-73) and docosatetraenoic acid (DTAn-6) (compounds 74-83), and the omega-3 counterpart of DPAn-6, docosapentaenoic acid (DPAn-3) (compounds 84-97). Further compounds are the docosanoids 98-115.

TABLE 1

| | |
|---|---|
| 10,17- Dihydroxy DPAn-6 58 | 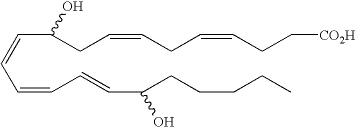 |
| 16,17- Dihydroxy DPAn-6 (59) | 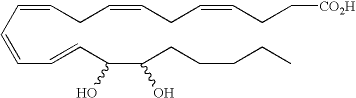 |
| 4,5 -Dihydroxy DPAn-6(60) | 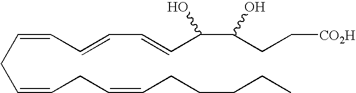 |
| 7,17- Dihydroxy DPAn-6 (61) | 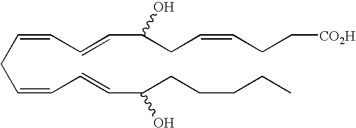 |
| 7-Hydroxy DPAn-6 (62) | 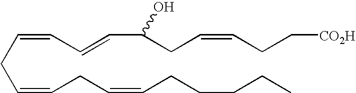 |
| 10- hydroxy DPAn-6 (63) | 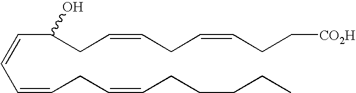 |
| 13- Hydroxy DPAn-6 (64) | 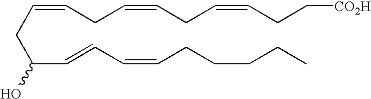 |
| 17- hydroxy DPAn-6 (65) | 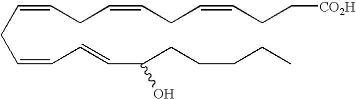 |
| 4,5,17- Trihydroxy DPAn-6 (66) | 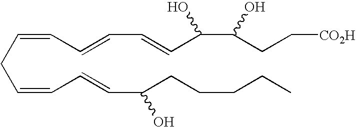 |
| 7,16,17- Trihydroxy DPAn-6 (67) | 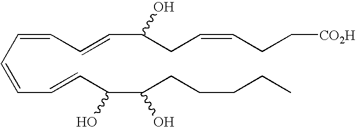 |
| 8-Hydroxy DPAn-6 (68) | 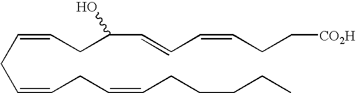 |
| 14-Hydroxy DPAn-6 (69) | 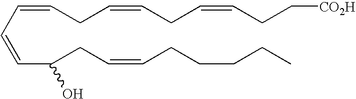 |
| 13,17- Dihydroxy DPAn-6 (70) | 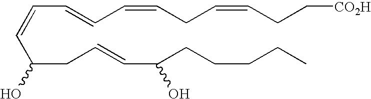 |
| 7,14- Dihydroxy DPAn-6 (71) | 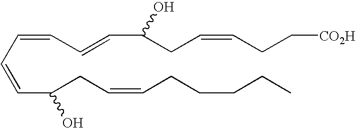 |
| 8,14- Dihydroxy DPAn-6 (72) | 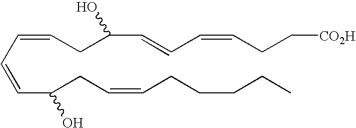 |
| 11- Hydroxy DPAn-6 (73) | 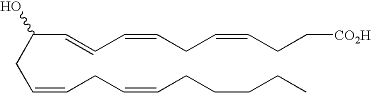 |
| 10,17-Di- hydroxy- DTAn-6 (74) | 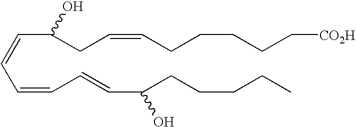 |
| 16,17-Di- hydroxy- DTAn-6 (75) | 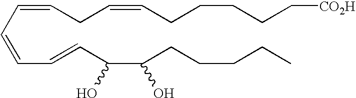 |
| 4,5-Di- hydroxy- DTAn-6 (76) | 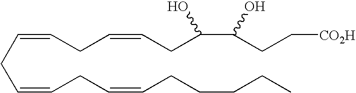 |
| 7,17-Di- hydroxy- DTAn-6 (77) | 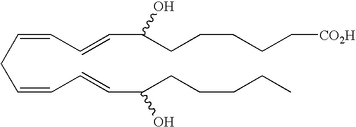 |
| 7- Hydroxy- DTAn-6 (78) | 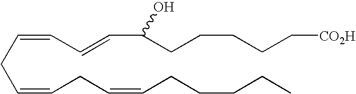 |
| 10- Hydroxy- DTAn-6 (79) | 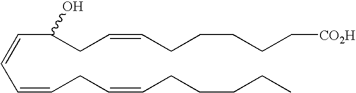 |
| 13- Hydroxy- DTAn-6 (80) | 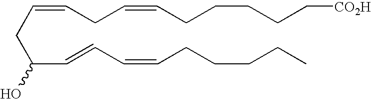 |
| 17- Hydroxy- DTAn-6 (81) | 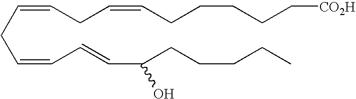 |
| 4,5,17- Trihy- droxy- DTAn-6 (82) | 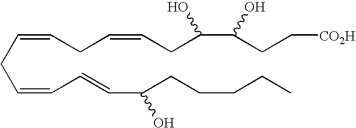 |
| 7,16,17- Trihy- droxy- DTAn-6 (83) | 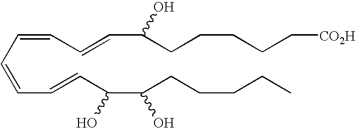 |
| 10,17-Di- hydroxy DPAn-3 (84) | 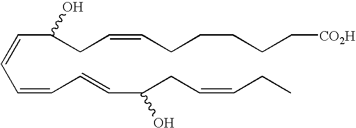 |
| 10,20-Di- hydroxy DPAn-3 (85) | 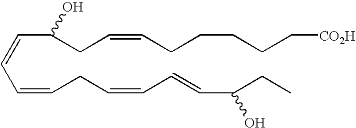 |
| 13,20-Di- hydroxy DPAn-3 (86) | 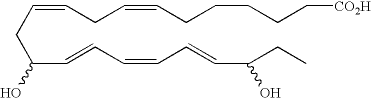 |
| 16,17-Di- hydroxy DPAn-3 (87) | 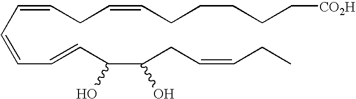 |
| 7,17-Di- hydroxy DPAn-3 (88) | 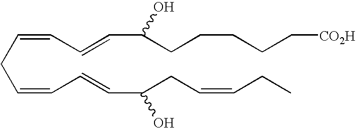 |
| 7-Hydroxy DPAn-3 (89) | 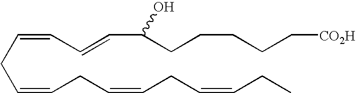 |
| 10- Hydroxy DPAn-3 (90) | 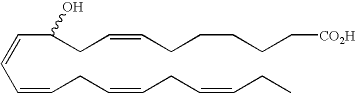 |
| 13- Hydroxy DPAn-3 (91) | 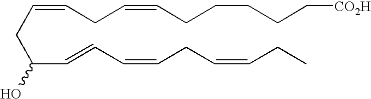 |
| 17- Hydroxy DPAn-3 (92) | 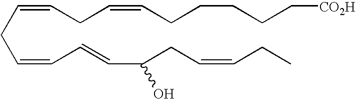 |
| 7,16,17- Trihy- droxy DPAn-3 (93) | 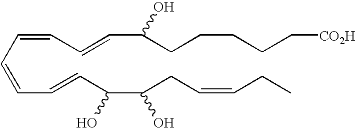 |
| 16- Hydroxy DPAn-3 (94) | 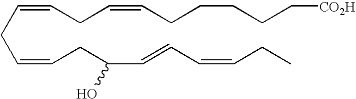 |
| 11- Hydroxy DPAn-3 (95) | 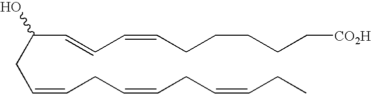 |
| 14- Hydroxy DPAn-3 (96) | 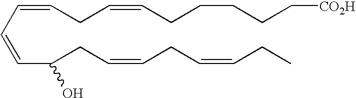 |
| 8,14-Di- hydroxy DPAn-3 (97) | 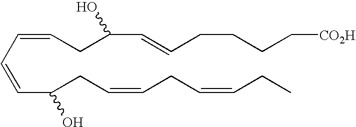 |
| 10,11- Epoxy DHA (98) | 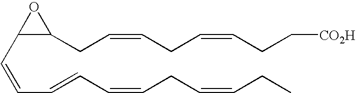 |
| 13,14-Di- hydroxy DHA (99) | 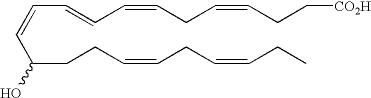 |
| 13,14- Epoxy DHA (100) | 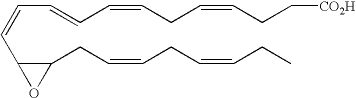 |
| 19,20- Epoxy DHA (191) | 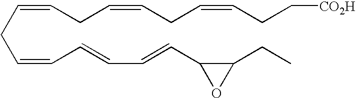 |
| 7,8-Epoxy DHA (102) | 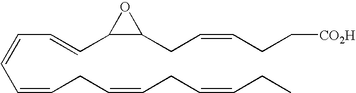 |
| 4,5- Epoxy-17- OH DPA 103) | 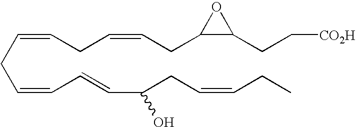 |
| 7,16,17- Trihy- droxy DTAn-3 (104) | 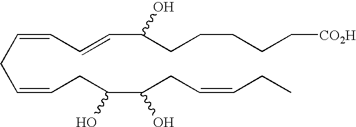 |
| 16,17-Di- hidroxy DTAn-3 (105) | 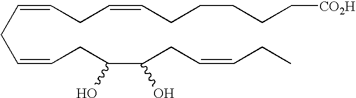 |
| 10,16,17- Trihy- droxy DTRAn-6 (106) | 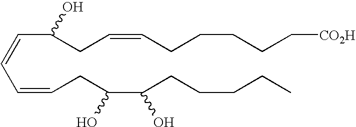 |
| 16,17-Di- hydroxy DTRAn-6 (107) | 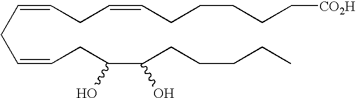 |
| 7,16,17- Trihy- droxy DTRAn-6 (108) | 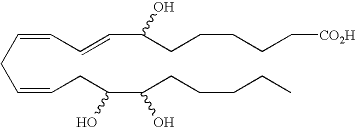 |
| 15-epi- lipoxin A4 (109) | 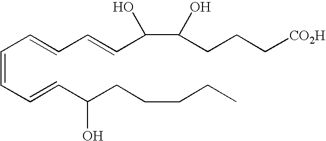 |
| 16,17- epoxy DHA (110) | 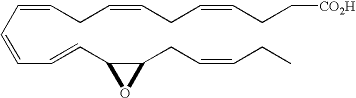 |
| 7,8-epoxy DPA (111) | 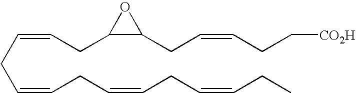 |
| 10,11 epoxy DPA (112) | 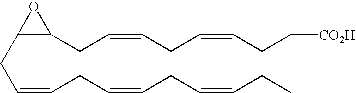 |
| 19,20 epoxy DPA (113) | 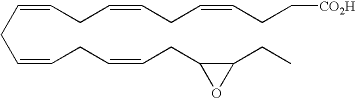 |
| 7-hydroxy DHA (114) | 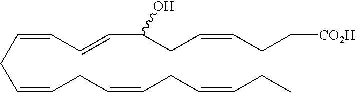 |
| 13,14 epoxy DPA (115) | 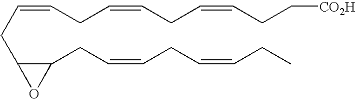 |

Other oxylipin compounds that are suitable for use in methods of the invention include analogs of the compounds shown in Table 1. Such compounds include but are not limited to those analogs wherein one or more double bonds are replaced by triple bonds, those wherein carboxy groups are derivatized to form esters, amides or salts, those wherein the hydroxyl-bearing carbons are further derivatized (with, for example, a substituted or unsubstituted, branched or unbranched alkyl, alkenyl, or alkynyl group, substituted or unsubstituted aryl group, substituted or unsubstituted, branched or unbranched alkylaryl group, halogen atom) to form tertiary alcohols (or ethers, esters, or other derivatives thereof), those wherein one or more hydroxyl groups are derivatized to form esters or protected alcohols, or those having combinations of any of the foregoing modifications.

Further oxylipin compounds suitable for use in methods of the invention include the following: isolated docosanoids of docosapentaenoic acid (DPAn-6); monohydroxy, dihydroxy, and trihydroxy derivatives of DPAn-6; isolated docosanoids of docosapentaenoic acid (DPAn-3); monohydroxy, dihydroxy, and trihydroxy derivatives of DPAn-3; isolated docosanoids of docosapentaenoic acid (DTAn-6); or monohydroxy, dihydroxy, and trihydroxy derivatives of DTAn-6.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

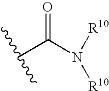

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

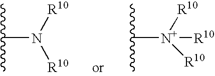

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

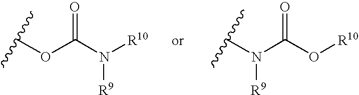

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

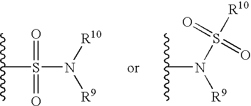

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰

wherein R¹⁰ represents a hydrocarbyl.

The term “thioether”, as used herein, is equivalent to an ether, wherein the oxygen is replaced with a sulfur.

The term “urea” is art-recognized and may be represented by the general formula

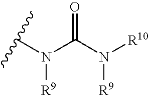

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPPS groups), glycol ethers, such as ethylene glycol and propylene glycol derivatives and allyl ethers.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms of the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “prodrug” refers to compounds that are rapidly transformed in vivo to yield a compound of formula 1, for example, by hydrolysis in blood. A thorough discussion is provided in T. Higuchi and V. Stella, “Pro-drugs as Novel Delivery Systems,” Vol. 14 of the A. C. S. Symposium Series, and in Bioreversible Carriers in Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. The prodrug may be designed to alter the metabolic stability or the transport characteristics of a compound, to mask side effects or toxicity, to improve the flavor of a compound or to alter other characteristics or properties of a compound. By virtue of knowledge of pharmacodynamic processes and drug metabolism in vivo, once a pharmaceutically active compound is identified, those of skill in the pharmaceutical art generally can design prodrugs of the compound (see, e.g., Nogrady (1985) Medicinal Chemistry A Biochemical Approach, Oxford University Press, N.Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

The synthesis of each of the compounds of formula A, compounds of any one of formulae 1 to 46, lipoxin compounds, or oxylipin compounds set forth above can be achieved by methods well-known in the art. For example, the synthesis of compounds of formula A or formulae 1 to 46 is set forth in US 2003/0191184, WO 2004/014835, WO 2004/078143, U.S. Pat. No. 6,670,396, US 2003/0236423 and US 2005/0228047, all of which are herein incorporated by reference. The synthesis of lipoxin compounds is set forth in US 2002/0107289, US 2004/0019110, US 2006/0009521, US 2005/0203184, US 2005/0113443. The preparation of oxylipin compounds is set forth in WO 2006/055965.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of formula A, compound of any one of formulae 1 to 46, lipoxin compound, oxylipin compound, or aspirin and/or an omega-3 fatty acid and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free. The excipients can be chosen, for example, to effect delayed release of an agent or to selectively target one or more cells, tissues or organs. The pharmaceutical composition can be in dosage unit form such as tablet, capsule, sprinkle capsule, granule, powder, syrup, suppository, injection or the like. The composition can also be present in a transdermal delivery system, e.g., a skin patch.

A pharmaceutically acceptable carrier can contain physiologically acceptable agents that act, for example, to stabilize or to increase the absorption of a compound such as a compound of formula A, compound of any one of formulae 1 to 46, lipoxin compound, oxylipin compound, or aspirin and/or an omega-3 fatty acid. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, boluses, powders, granules, pastes for application to the tongue); sublingually; anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein. The most preferred route of administration is the oral route.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of formula A, a compound of any one of formulae 1 to 46, a lipoxin compound, an oxylipin compound, or aspirin and/or an omega-3 fatty acid, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, the suitable daily dose of a compound of formula A, a compound of any one of formulae 1 to 46, a lipoxin compound, an oxylipin compound, or a combination of aspirin and an omega-3 fatty acid for treating mucositis will be 2 times, 5 times, 10 times, or 20 times more than the dose administered for treating inflammation. In certain embodiments, the suitable daily dose of a compound of formula A, a compound of any one of formulae 1 to 46, a lipoxin compound, an oxylipin compound, or a combination of aspirin and an omega-3 fatty acid for treating mucositis will be 2 times, 5 times, 10 times, or 20 times less than the dose administered for treating inflammation.

In certain embodiments, compounds of formula A, compounds of any one of formulae 1 to 46, lipoxin compounds, oxylipin compounds, or a combination of aspirin and an omega-3 fatty acid may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

In one embodiment, the method of treating mucositis may comprise administering a compound of formula A, compound of any one of formulae 1 to 46, lipoxin compound, or oxylipin compound, or a combination of aspirin and an omega-3 fatty acid conjointly with an additional agent useful in the treatment of mucositis. In certain embodiments, the compound of formula A, compound of any one of formulae 1 to 46, lipoxin compound, or oxylipin compound, or the combination of aspirin and an omega-3 fatty acid may be conjointly administered with an antimicrobial agent. In certain embodiments, the compound of formula A, compound of any one of formulae 1 to 46, lipoxin compound, or oxylipin compound, or the combination of aspirin and an omega-3 fatty acid may be conjointly administered with a growth factor. In certain embodiments, the compound of formula A, compound of any one of formulae 1 to 46, lipoxin compound, or oxylipin compound, or the combination of aspirin and an omega-3 fatty acid may be conjointly administered with an agent that inhibits the synthesis of ceramide, an agent that blocks the activity of ceramide, or an agent that degrades ceramide.

In one embodiment, the method of treating mucositis may comprise administering a compound of formula A, compound of any one of formulae 1 to 46, lipoxin compound, or oxylipin compound, or a combination of aspirin and an omega-3 fatty acid conjointly with a chemotherapeutic agent. Chemotherapeutic agents that may be conjointly administered with compounds of formula A, compounds of any one of formulae 1 to 46, lipoxin compounds, oxylipin compounds, or a combination of aspirin and an omega-3 fatty acid include: aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

Many combination therapies have been developed for the treatment of cancer. In certain embodiments, compounds of formula A, compounds of any one of formulae 1 to 46, lipoxin compounds, oxylipin compounds, or a combination of aspirin and an omega-3 fatty acid may be conjointly administered with a combination therapy. Examples of combination therapies with which compounds of formula A, compounds of any one of formulae 1 to 46, lipoxin compounds, oxylipin compounds, or a combination of aspirin and an omega-3 fatty acid may be conjointly administered are included in Table 2.

TABLE 2
Exemplary combinatorial therapies for the treatment of cancer.

| Name | Therapeutic agents |
|---|---|
| ABV | Doxorubicin, Bleomycin, Vinblastine |
| ABVD | Doxorubicin, Bleomycin, Vinblastine, Dacarbazine |
| AC (Breast) | Doxorubicin, Cyclophosphamide |
| AC (Sarcoma) | Doxorubicin, Cisplatin |
| AC (Neuroblastoma) | Cyclophosphamide, Doxorubicin |
| ACE | Cyclophosphamide, Doxorubicin, Etoposide |
| ACe | Cyclophosphamide, Doxorubicin |
| AD | Doxorubicin, Dacarbazine |
| AP | Doxorubicin, Cisplatin |
| ARAC-DNR | Cytarabine, Daunorubicin |
| B-CAVe | Bleomycin, Lomustine, Doxorubicin, Vinblastine |
| BCVPP | Carmustine, Cyclophosphamide, Vinblastine, |
| | Procarbazine, Prednisone |
| BEACOPP | Bleomycin, Etoposide, Doxorubicin, |
| | Cyclophosphamide, Vincristine, Procarbazine, |
| | Prednisone, Filgrastim |
| BEP | Bleomycin, Etoposide, Cisplatin |
| BIP | Bleomycin, Cisplatin, Ifosfamide, Mesna |
| BOMP | Bleomycin, Vincristine, Cisplatin, Mitomycin |
| CA | Cytarabine, Asparaginase |
| CABO | Cisplatin, Methotrexate, Bleomycin, Vincristine |
| CAF | Cyclophosphamide, Doxorubicin, Fluorouracil |
| CAL-G | Cyclophosphamide, Daunorubicin, Vincristine, |
| | Prednisone, Asparaginase |
| CAMP | Cyclophosphamide, Doxorubicin, Methotrexate, |
| | Procarbazine |
| CAP | Cyclophosphamide, Doxorubicin, Cisplatin |
| CaT | Carboplatin, Paclitaxel |
| CAV | Cyclophosphamide, Doxorubicin, Vincristine |
| CAVE ADD | CAV and Etoposide |
| CA-VP16 | Cyclophosphamide, Doxorubicin, Etoposide |
| CC | Cyclophosphamide, Carboplatin |
| CDDP/VP-16 | Cisplatin, Etoposide |
| CEF | Cyclophosphamide, Epirubicin, Fluorouracil |
| CEPP(B) | Cyclophosphamide, Etoposide, Prednisone, with or |
| | without/Bleomycin |
| CEV | Cyclophosphamide, Etoposide, Vincristine |
| CF | Cisplatin, Fluorouracil or Carboplatin Fluorouracil |
| CHAP | Cyclophosphamide or Cyclophosphamide, Altretamine, |
| | Doxorubicin, Cisplatin |
| ChlVPP | Chlorambucil, Vinblastine, Procarbazine, Prednisone |
| CHOP | Cyclophosphamide, Doxorubicin, Vincristine, |
| | Prednisone |
| CHOP-BLEO | Add Bleomycin to CHOP |
| CISCA | Cyclophosphamide, Doxorubicin, Cisplatin |
| CLD-BOMP | Bleomycin, Cisplatin, Vincristine, Mitomycin |
| CMF | Methotrexate, Fluorouracil, Cyclophosphamide |
| CMFP | Cyclophosphamide, Methotrexate, Fluorouracil, |
| | Prednisone |
| CMFVP | Cyclophosphamide, Methotrexate, Fluorouracil, |
| | Vincristine, Prednisone |
| CMV | Cisplatin, Methotrexate, Vinblastine |
| CNF | Cyclophosphamide, Mitoxantrone, Fluorouracil |
| CNOP | Cyclophosphamide, Mitoxantrone, Vincristine, |
| | Prednisone |
| COB | Cisplatin, Vincristine, Bleomycin |
| CODE | Cisplatin, Vincristine, Doxorubicin, Etoposide |
| COMLA | Cyclophosphamide, Vincristine, Methotrexate, |
| | Leucovorin, Cytarabine |
| COMP | Cyclophosphamide, Vincristine, Methotrexate, |
| | Prednisone |
| Cooper Regimen | Cyclophosphamide, Methotrexate, Fluorouracil, |
| | Vincristine, Prednisone |
| COP | Cyclophosphamide, Vincristine, Prednisone |
| COPE | Cyclophosphamide, Vincristine, Cisplatin, Etoposide |
| COPP | Cyclophosphamide, Vincristine, Procarbazine, |
| | Prednisone |
| CP(Chronic lymphocytic | Chlorambucil, Prednisone |
| leukemia) | |
| CP (Ovarian Cancer) | Cyclophosphamide, Cisplatin |
| CT | Cisplatin, Paclitaxel |
| CVD | Cisplatin, Vinblastine, Dacarbazine |
| CVI | Carboplatin, Etoposide, Ifosfamide, Mesna |
| CVP | Cyclophosphamide, Vincristine, Prednisome |
| CVPP | Lomustine, Procarbazine, Prednisone |
| CYVADIC | Cyclophosphamide, Vincristine, Doxorubicin, |
| | Dacarbazine |
| DA | Daunorubicin, Cytarabine |
| DAT | Daunorubicin, Cytarabine, Thioguanine |
| DAV | Daunorubicin, Cytarabine, Etoposide |
| DCT | Daunorubicin, Cytarabine, Thioguanine |
| DHAP | Cisplatin, Cytarabine, Dexamethasone |
| DI | Doxorubicin, Ifosfamide |
| DTIC/Tamoxifen | Dacarbazine, Tamoxifen |
| DVP | Daunorubicin, Vincristine, Prednisone |
| EAP | Etoposide, Doxorubicin, Cisplatin |
| EC | Etoposide, Carboplatin |
| EFP | Etoposie, Fluorouracil, Cisplatin |
| ELF | Etoposide, Leucovorin, Fluorouracil |
| EMA 86 | Mitoxantrone, Etoposide, Cytarabine |
| EP | Etoposide, Cisplatin |
| EVA | Etoposide, Vinblastine |
| FAC | Fluorouracil, Doxorubicin, Cyclophosphamide |
| FAM | Fluorouracil, Doxorubicin, Mitomycin |
| FAMTX | Methotrexate, Leucovorin, Doxorubicin |
| FAP | Fluorouracil, Doxorubicin, Cisplatin |
| F-CL | Fluorouracil, Leucovorin |
| FEC | Fluorouracil, Cyclophosphamide, Epirubicin |
| FED | Fluorouracil, Etoposide, Cisplatin |
| FL | Flutamide, Leuprolide |
| FZ | Flutamide, Goserelin acetate implant |
| HDMTX | Methotrexate, Leucovorin |
| Hexa-CAF | Altretamine, Cyclophosphamide, Methotrexate, |
| | Fluorouracil |
| ICE-T | Ifosfamide, Carboplatin, Etoposide, Paclitaxel, Mesna |
| IDMTX/6-MP | Methotrexate, Mercaptopurine, Leucovorin |
| IE | Ifosfamide, Etoposie, Mesna |
| IfoVP | Ifosfamide, Etoposide, Mesna |
| IPA | Ifosfamide, Cisplatin, Doxorubicin |
| M-2 | Vincristine, Carmustine, Cyclophosphamide, |
| | Prednisone, Melphalan |
| MAC-III | Methotrexate, Leucovorin, Dactinomycin, |
| | Cyclophosphamide |
| MACC | Methotrexate, Doxorubicin, Cyclophosphamide, |
| | Lomustine |
| MACOP-B | Methotrexate, Leucovorin, Doxorubicin, |
| | Cyclophosphamide, Vincristine, Bleomycin, Prednisone |
| MAID | Mesna, Doxorubicin, Ifosfamide, Dacarbazine |
| m-BACOD | Bleomycin, Doxorubicin, Cyclophosphamide, |
| | Vincristine, Dexamethasone, Methotrexate, Leucovorin |
| MBC | Methotrexate, Bleomycin, Cisplatin |
| MC | Mitoxantrone, Cytarabine |
| MF | Methotrexate, Fluorouracil, Leucovorin |
| MICE | Ifosfamide, Carboplatin, Etoposide, Mesna |
| MINE | Mesna, Ifosfamide, Mitoxantrone, Etoposide |
| mini-BEAM | Carmustine, Etoposide, Cytarabine, Melphalan |
| MOBP | Bleomycin, Vincristine, Cisplatin, Mitomycin |
| MOP | Mechlorethamine, Vincristine, Procarbazine |
| MOPP | Mechlorethamine, Vincristine, Procarbazine, Prednisone |
| MOPP/ABV | Mechlorethamine, Vincristine, Procarbazine, |
| | Prednisone, Doxorubicin, Bleomycin, Vinblastine |
| MP (multiple myeloma) | Melphalan, Prednisone |
| MP (prostate cancer) | Mitoxantrone, Prednisone |
| MTX/6-MO | Methotrexate, Mercaptopurine |
| MTX/6-MP/VP | Methotrexate, Mercaptopurine, Vincristine, Prednisone |
| MTX-CDDPAdr | Methotrexate, Leucovorin, Cisplatin, Doxorubicin |
| MV (breast cancer) | Mitomycin, Vinblastine |
| MV (acute myelocytic | Mitoxantrone, Etoposide |
| leukemia) | |
| M-VAC Methotrexate | Vinblastine, Doxorubicin, Cisplatin |
| MVP Mitomycin | Vinblastine, Cisplatin |
| MVPP | Mechlorethamine, Vinblastine, Procarbazine, Prednisone |
| NFL | Mitoxantrone, Fluorouracil, Leucovorin |
| NOVP | Mitoxantrone, Vinblastine, Vincristine |
| OPA | Vincristine, Prednisone, Doxorubicin |
| OPPA | Add Procarbazine to OPA. |
| PAC | Cisplatin, Doxorubicin |
| PAC-I | Cisplatin, Doxorubicin, Cyclophosphamide |
| PA-CI | Cisplatin, Doxorubicin |
| PC | Paclitaxel, Carboplatin or Paclitaxel, Cisplatin |
| PCV | Lomustine, Procarbazine, Vincristine |
| PE | Paclitaxel, Estramustine |
| PFL | Cisplatin, Fluorouracil, Leucovorin |
| POC | Prednisone, Vincristine, Lomustine |
| ProMACE | Prednisone, Methotrexate, Leucovorin, Doxorubicin, |
| | Cyclophosphamide, Etoposide |
| ProMACE/cytaBOM | Prednisone, Doxorubicin, Cyclophosphamide, |
| | Etoposide, Cytarabine, Bleomycin, Vincristine, |
| | Methotrexate, Leucovorin, Cotrimoxazole |
| PRoMACE/MOPP | Prednisone, Doxorubicin, Cyclophosphamide, |
| | Etoposide, Mechlorethamine, Vincristine, Procarbazine, |
| | Methotrexate, Leucovorin |
| Pt/VM | Cisplatin, Teniposide |
| PVA | Prednisone, Vincristine, Asparaginase |
| PVB | Cisplatin, Vinblastine, Bleomycin |
| PVDA | Prednisone, Vincristine, Daunorubicin, Asparaginase |
| SMF | Streptozocin, Mitomycin, Fluorouracil |
| TAD | Mechlorethamine, Doxorubicin, Vinblastine, |
| | Vincristine, Bleomycin, Etoposide, Prednisone |
| TCF | Paclitaxel, Cisplatin, Fluorouracil |
| TIP | Paclitaxel, Ifosfamide, Mesna, Cisplatin |
| TTT | Methotrexate, Cytarabine, Hydrocortisone |
| Topo/CTX | Cyclophosphamide, Topotecan, Mesna |
| VAB-6 | Cyclophosphamide, Dactinomycin, Vinblastine, |
| | Cisplatin, Bleomycin |
| VAC | Vincristine, Dactinomycin, Cyclophosphamide |
| VACAdr | Vincristine, Cyclophosphamide, Doxorubicin, |
| | Dactinomycin, Vincristine |
| VAD | Vincristine, Doxorubicin, Dexamethasone |
| VATH | Vinblastine, Doxorubicin, Thiotepa, Flouxymesterone |
| VBAP | Vincristine, Carmustine, Doxorubicin, Prednisone |
| VBCMP | Vincristine, Carmustine, Melphalan, Cyclophosphamide, |
| | Prednisone |
| VC | Vinorelbine, Cisplatin |
| VCAP | Vincristine, Cyclophosphamide, Doxorubicin, |
| | Prednisone |
| VD | Vinorelbine, Doxorubicin |
| VelP | Vinblastine, Cisplatin, Ifosfamide, Mesna |
| VIP | Etoposide, Cisplatin, Ifosfamide, Mesna |
| VM | Mitomycin, Vinblastine |
| VMCP | Vincristine, Melphalan, Cyclophosphamide, Prednisone |
| VP | Etoposide, Cisplatin |
| V-TAD | Etoposide, Thioguanine, Daunorubicin, Cytarabine |
| 5 + 2 | Cytarabine, Daunorubicin, Mitoxantrone |
| 7 + 3 | Cytarabine with/, Daunorubicin or Idarubicin or |
| | Mitoxantrone |
| “8 in 1” | Methylprednisolone, Vincristine, Lomustine, |
| | Procarbazine, Hydroxyurea, Cisplatin, Cytarabine, |
| | Dacarbazine |

In certain embodiments, the present invention provides a kit comprising: a) one or more single dosage forms of a compound of formula A, compound of any one of formulae 1 to 46, lipoxin compound, or oxylipin compound, or a combination of aspirin and an omega-3 fatty acid; b) one or more single dosage forms of a chemotherapeutic agent as mentioned above; and c) instructions for the administration of the compound of formula A, compound of any one of formulae 1 to 46, lipoxin compound, or oxylipin compound, or the combination of aspirin and an omega-3 fatty acid and the chemotherapeutic agent.

In certain embodiments, a compound of formula A, compound of any one of formulae 1 to 46, lipoxin compound, oxylipin compound, or a combination of aspirin and an omega-3 fatty acid may be conjointly administered with non-chemical methods of cancer treatment. In certain embodiments, a compound of formula A, compound of any one of formulae 1 to 46, lipoxin compound, oxylipin compound, or a combination of aspirin and an omega-3 fatty acid may be conjointly administered with radiation therapy. In certain embodiments, a compounds of formula A, compound of any one of formulae 1 to 46, lipoxin compound, oxylipin compound, or a combination of aspirin and an omega-3 fatty acid may be conjointly administered with surgery, with thermoablation, with focused ultrasound therapy, or with cryotherapy.

In certain embodiments, different compounds of formula A, compounds of any one of formulae 1 to 46, lipoxin compounds, or oxylipin compounds may be conjointly administered with one another, and such combinations may be conjointly administered with other therapeutics as discussed above. In certain embodiments, different compounds of formula A, compounds of′ any one of formulae 1 to 46, lipoxin compounds, or oxylipin compounds may be conjointly administered with a combination of aspirin and an omega-3 fatty acid, and such combinations may be conjointly administered with other therapeutics as discussed above.

In embodiments where a combination of aspirin and an omega-3 fatty acid are administered, the aspirin and omega-3 fatty acid can be administered simultaneously, e.g., as a single formulation comprising both components or in separate formulations, or can be administered at separate times, provided that, at least at certain times during the therapeutic regimen, both the aspirin and omega-3 fatty acid are present simultaneously in the patient at levels that allow the omega-3 fatty acid to be metabolized as described in Serhan, et. al., 2002, J. Exp. Med., 196: 1025-1037. In certain such embodiments, the omega-3 fatty acid is provided in the form of a partially purified natural extract, such as fish oil, while in other embodiments, the omega-3 fatty acid may be provided as a substantially pure preparation of one or more omega-3 fatty acids, such as a C18:3, C20:5, or C22:6 fatty acid, particularly eicosapentaenoic acid or docosahexaenoic acid. A substantially pure preparation of one or more omega-3 fatty acids refers to a composition wherein the fatty acid component is at least 90%, at least 95%, or even at least 98% of one or more omega-3 fatty acids, such as one or more specified omega-3 fatty acids. Non-fatty acid components, such as excipients or other materials added during formulation, are not considered for the purpose of determining whether the fatty acid component meets the desired level of purity.

In certain embodiments, a COX-2 inhibitor other than aspirin, such as celecoxib, rofecoxib, valdecoxib, lumiracoxib, etoricoxib, NS-398, or parecoxib, may be used in combination with an omega-3 fatty acid for the treatment or prevention of mucositis in any of the various embodiments discussed herein. The combination of different COX-2 inhibitors with an omega-3 fatty acid may result in the production of different subsets or proportions of active omega-3 metabolites.

This invention includes the use of pharmaceutically acceptable salts of compounds of formula A, compounds of any one of formulae 1 to 46, lipoxin compounds, or oxylipin compounds in the compositions and methods of the present invention. In certain embodiments, contemplated salts of the invention include alkyl, dialkyl, trialkyl or tetra-alkyl ammonium salts. In certain embodiments, contemplated salts of the invention include Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

### Examples

### Example 1

### A Study of the Efficacy of Compound X in the Treatment of Oral Mucositis Induced by Acute Radiation in Hamsters

### Study Objective:

The objective of this study was to evaluate the effect of Compound X,

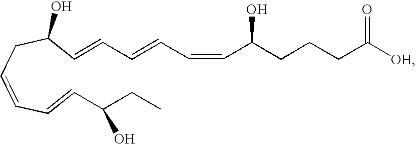

administered by intra-peritoneal injection on the frequency, severity and duration of oral mucositis in hamsters induced by acute radiation.

### Materials and Methods:

| | |
|---|---|
| Species/strain: | Golden Syrian Hamster/LVG |
| Physiological state: | Normal |
| Age/weight range at | Animals aged 5 to 6 weeks with body weight |
| start of study: | of approximately 90 g |
| Animal supplier: | Charles River Laboratories |
| Number/sex of animals: | 32 male |
| Randomization: | Animals were randomly and prospectively |
| | divided into four (4) treatment groups of eight |
| | (8) animals each prior to treatment or |
| | irradiation. |

### Administration of Test Article:

| | |
|---|---|
| Route and method of | Intra-peritoneal injection |
| administration: | |
| Frequency and duration | Once daily and one group twice daily |
| of dosing: | |
| Administered doses: | 0.5, 5 and 50 ug/kg QD and 5 ug/kg BID |
| Administered volume(s): | 5 ml/kg (~0.45 ml/hamster) |

### Experimental Design:

Thirty-two (32) Syrian Golden Hamsters were given an acute radiation dose of 40 Gy directed to their left buccal cheek pouch. This was accomplished by anesthetizing the animals and everting the left buccal pouch, while protecting the rest of the animals with a lead shield. Test material was given i.p daily or twice daily as detailed in Table 3. Dosing began one days before radiation (day −1) and continued until day 15, including the day of radiation (day 0, 30 minutes before radiation), Mucositis was evaluated clinically starting on Day 6, and continued on alternate days until day 28.

TABLE 3
Study Design

| Group | Number of | | Treatment | Volume |
|---|---|---|---|---|
| Number | Animals | Treatment | Schedule* | (mL) |
| 1 | 8 males | Vehicle Control | Daily | 5 ml/kg |
| | | | Day −1 to 15 | |
| 2 | 8 males | Compound X | Daily | 5 ml/kg |
| | | 0.5 ug/kg | Day −1 to 15 | |
| 3 | 8 males | Compound X | Daily | 5 ml/kg |
| | | 5 ug/kg | Day −1 to 15 | |
| 4 | 8 males | Compound X | Daily | 5 ml/kg |
| | | 50 ug/kg | Day −1 to 15 | |
| 5 | 8 males | Compound X | BID | 5 ml/kg |
| | | 5 ug/kg | Day −1 to 15 | |

### Mucositis Induction:

Mucositis was induced using a single dose of radiation (40 Gy/dose) administered to all animals on Day 0. Radiation was generated with a 160 kilovolt potential (18.75-ma) source at a focal distance of 21 cm, hardened with a 3.0 mm Al filtration system. Irradiation targeted the left buccal pouch mucosa at a rate of 1.32 Gy/minute. Prior to irradiation, animals were anesthetized with an intraperitoneal injection of ketamine (160 mg/kg) and xylazine (8 mg/kg). The left buccal pouch was everted, fixed and isolated using a lead shield.

### Mucositis Scoring:

Starting on day 6 and continuing every second day thereafter (days 8, 10, 12, 14, 16, 18, 20, 22, 24, 26, & 28), each animal was photographed and evaluated for mucositis scoring. Parameters to be measured included the mucositis score, weight change and survival. For the evaluation of mucositis, the animals were anesthetized with inhalation anesthetics, and the left pouch everted. Mucositis was scored visually by comparison to a validated photographic scale, ranging from 0 for normal, to 5 for severe ulceration (clinical scoring). In descriptive terms, this scale was defined as follows:

| Score: | Description: |
|---|---|
| 0 | Pouch completely healthy. No erythema or vasodilation. |
| 1 | Light to severe erythema and vasodilation. No erosion of mucosa. |
| 2 | Severe erythema and vasodilation. Erosion of superficial aspects of |
| | mucosa leaving denuded areas. Decreased stippling of mucosa. |
| 3 | Formation of off-white ulcers in one or more places. Ulcers may have a |
| | yellow/gray due to pseudomembrane. Cumulative size of ulcers should |
| | equal about ¼ of the pouch. Severe erythema and vasodilation. |
| 4 | Cumulative seize of ulcers should equal about ½ of the pouch. Loss of |
| | pliability. Severe erythema and vasodilation. |
| 5 | Virtually all of pouch is ulcerated. Loss of pliability (pouch can only |
| | partially be extracted from mouth) |

A score of 1-2 was considered to represent a mild stage of the disease, whereas a score of 3-5 was considered to indicate moderate to severe mucositis. Following this preliminary clinical scoring, a photograph was taken of each animal's mucosa using a standardized technique. At the conclusion of the experiment, film was developed and the photographs randomly numbered for blinded scoring. Thereafter, two independent, trained observers graded the photographs in blinded fashion using the above-described scale. For each photograph the actual blinded score was based upon the average of the evaluator's scores. Only the scores from this blinded, photographic evaluation were statistically analyzed and reported in the final study report.

### Mucositis Evaluation:

Using the blinded photographs, the grade of mucositis was scored, beginning day 6, and for every second day thereafter, through and including day 28. The effect on mucositis of each drug treatment compared to vehicle control was assessed according to the following parameters:

### The Difference in the Number of Days Hamsters in Each Group Have Severe (Score ≧3) Mucositis.

On each day the animals were scored (evaluation day), the number of animals with a blinded mucositis score of ≧3 in each drug treatment group was compared to the vehicle control group. Differences were analyzed on a daily as well as a cumulative basis. Treatment success was considered a statistically significantly lower number of hamsters with this score in a drug treatment group, versus control as determined by chi-square analysis.

### The Rank Sum Differences in Daily Mucositis Scores.

For each evaluation day the scores of the vehicle control group was compared to those of the treated groups using the non-parametric rank sum analysis. Treatment success was considered as a statistically significant lowering of scores in the treated group on 2 or more days from day 6 to day 28.

To evaluate the effect of test agents on mucositis resolution, the time to healing was compared between test and controls. Resolution was defined as the absence of ulcerative lesions (scores <3).

### Body Weight:

Every day for the period of the study, each animal was weighed and its survival recorded, in order to assess possible differences in animal weight among treatment groups as an indication for mucositis severity and/or possible toxicity resulting from the treatments.

### Data Analysis and Reporting:

Statistical differences between treatment groups were determined using Student's t-test, Mann-Whitney U test and Chi-square analysis with a critical value of 0.05. It was anticipated that up to 10% animal death may occur, primarily as a result of the administration of anesthetics. However, the number of animals expected to remain alive at Day 28 (6 per treatment group) was considered acceptable for statistical evaluation.

FIG. 1 shows the Chi-squared analysis of days with a score of 3 or higher after administration of compound X versus control. The data shows, inter alia, the reduction of days with a clinical score of 3 or higher for the 5 ug/kg and 50 ug/kg dose groups as compared to control. The 5 ug/kg and 50 ug/kg dose groups show a 40% and 30% reduction, respectively, as compared to control.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of inhibiting the development of mucositis in a patient receiving chemotherapy or radiation therapy, comprising administering to said patient a compound of formula A, compound of any one of formulae 1 to 46, lipoxin compound, or oxylipin compound.

2. The method of claim 1, wherein said compound of formula A, compound of any one of formulae 1 to 46, lipoxin compound, or oxylipin compound is administered conjointly with said chemotherapy or radiation therapy.

3. A method of treating mucositis in a patient, comprising administering to said patient a compound of formula A, compound of any one of formulae 1 to 46, lipoxin compound, or oxylipin compound.

4. A method of promoting survival of a patient receiving a therapeutic regimen associated with an increased risk of mucositis, comprising administering to said patient a compound of formula A, compound of any one of formulae 1 to 46, lipoxin compound, or oxylipin compound.

5. The method of claim 4, wherein said compound of formula A, compound of any one of formulae 1 to 46, lipoxin compound, or oxylipin compound is administered conjointly with said therapeutic regimen.

6. A method of treating ulceration or necrosis of mucosal tissue in a patient receiving chemotherapy or radiation therapy, comprising administering to said patient a compound of formula A, compound of any one of formulae 1 to 46, lipoxin compound, or oxylipin compound.

7. The method according to any one of claims 1 to 6, wherein the compound of formula A, compound of any one of formulae 1 to 46, lipoxin compound, or oxylipin compound is selected from a compound of any one of Formulae 1 to 46 or 50 to 115.

8. The method according to any one of claims 1 to 6, wherein the compound is Compound X, 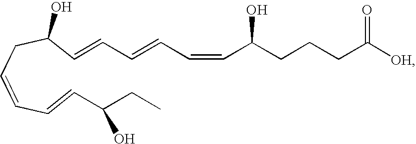
or a pharmaceutically acceptable salt thereof.

9-14. (canceled)

15. A compound of formula 45, 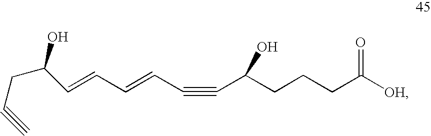
or a pharmaceutically acceptable salt thereof.

16. A pharmaceutical composition comprising a compound according to claim 15, or a pharmaceutically acceptable salt thereof, and a pharmaceutically acceptable carrier.
